(19) United States
(12) Patent Application Publication
(10) Pub. No.: US 2026/0108705 A1
(43) Pub. Date: Apr. 23, 2026

(54) STEERABLE CATHETERS

(71) Applicants: AORTYX S.L., Teia (ES); INSTITUT QUÍMIC DE SARRIA CETS FUNDACIÓ, Barcelona (ES); HOSPITAL CLÍNIC DE BARCELONA, Barcelona (ES)

(72) Inventors: Carlota Molhoek Baleri, Teia (ES); Alejandro Aranda Gil, Teia (ES); Noemí Balà Palasí, Teia (ES); Vicente Riambau Alonso, Teia (ES); Jordi Martorell López, Teia (ES)

(73) Assignees: AORTYX S.L., Teia (ES); INSTITUT QUÍMIC DE SARRIA CETS FUNDACIÓ, Barcelona (ES); HOSPITAL CLÍNIC DE BARCELONA, Barcelona (ES)

(21) Appl. No.: 19/307,624

(22) Filed: Aug. 22, 2025

Related U.S. Application Data
(63) Continuation of application No. PCT/EP2024/054855, filed on Feb. 26, 2024.

(30) Foreign Application Priority Data
Feb. 24, 2023 (EP) 23382175

(51) Int. Cl. A61M 25/01; A61F 2/24

(52) U.S. Cl. CPC A61M 25/0147; A61F 2/2481; A61F 2002/2484

(57) ABSTRACT

This present disclosure relates to steerable catheters comprising first and second steering sections that are respectively configured to steer in opposite directions within the same plane. The present disclosure also relates to a system comprising a steerable catheter according to the present disclosure and a patch deployment device that is deployable via the catheter.

### CROSS-REFERENCE TO RELATED APPLICATIONS

The present disclosure is filed under 35 U.S.C. § 111(a) and claims the benefit of priority under 35 U.S.C. § 365(c) to International Patent Application No. PCT/EP2024/054855, entitled “STEERABLE CATHETERS” and filed Feb. 26, 2024, and which claims the benefit of priority to European Patent Application No. EP23382175, entitled “STEERABLE CATHETERS” and filed Feb. 24, 2023, the entire contents of both are incorporated by reference herein.

### FIELD

The present disclosure relates to steerable catheters comprising first and second steering sections that are respectively configured to steer in opposite directions such as to create free space adjacent to an anatomical defect requiring surgical intervention. The steerable catheters of the present disclosure are particularly adapted for use in medical methods that require an intervention with an interior wall of an anatomical lumen, for example the deployment of a cardiovascular repair patch onto a cardiovascular wall. The present disclosure also relates to a system for deploying a patch to an anatomical lumen, the system comprising a steerable catheter according to the present disclosure and a patch deployment device that is deployable via the catheter. The present disclosure also relates to a method of applying a patch to a defect site in an anatomical lumen using the system of the present disclosure.

### BACKGROUND

Catheters are used in a variety of medical procedures that require access to the anatomical lumina (e.g. blood vessels), for example for the deployment of a medical device. To address this need, a wide variety of catheters have been developed, ranging from basic delivery tubes to more complex devices having a range of functionalities.

A problem commonly encountered in such medical procedures is the need for a catheter to navigate a tortuous path to access a treatment site. Examples are navigation to the left side of the heart through the atrial septum or navigation to the right ventricle via the tricuspid valve. A range of catheters have been developed that are able to adopt non-linear configurations that enable the catheter to navigate in tortuous vasculature, steer through tight curves, and access hard-to-reach sites.

A first category of catheters contain flexible sections that are able to passively deflect when the catheter comes into contact with, for example, a vascular wall. A second category of catheters contain shape-set sections that adopt, for example, a curved shape in the absence of any external forces. The present disclosure relates to a third category of catheters; the steerable catheters. Steerable catheters, as the term is used herein, are catheters in which one or more sections of the catheter can be actively controlled to modify their shape, for instance to assist in navigating a tortuous path through an anatomical lumen.

It has been proposed to use cardiovascular repair patches to repair damaged vascular tissue. For example, WO 2019/175288 discloses a vascular repair patch that can be used for the repair of damage to the walls of blood vessels, in particular for the repair of aortic dissection. Aortic dissection is a life-threatening condition caused by a tear in the intimal layer of the aorta or bleeding within the aortic wall, resulting in the separation (dissection) of the layers of the aortic wall. Blood flow forces the layers of aortic wall apart, resulting in the formation of a channel, known as a false lumen, between the layers. The condition can be fatal if the blood-filled channel ruptures through the outside of the aortic wall.

Endoluminal techniques are a minimally invasive method for accessing and repairing defects in anatomical lumina. WO 2021/058602 discloses an endovascular method of deploying a vascular repair patch using a catheter in combination with a deployment device which is deployable via the catheter. The deployment device adopts an unexpanded configuration when constrained within the catheter lumen, but opens to an expanded configuration when advanced from the distal opening of the catheter lumen with the aid of a push wire. A patch is attached to the deployment device and unfolds ready for application to the vascular defect when the deployment device adopts the expanded configuration. Once the patch is secured to the treatment site, the deployment device can be retracted into the catheter, whereupon it readopts the unexpanded configuration, and the catheter may then be removed from the patient.

One problem that must be addressed when deploying a cardiovascular repair patch in this way is that the site of treatment (being a side wall of a blood vessel) is generally not aligned with the axial direction of the catheter. Unlike in other endovascular methods employing catheters, it is not sufficient merely to be able to navigate through the vasculature. Instead, the tip of the catheter must be actively steered to face the cardiovascular wall so that the patch is deployed effectively. WO2021/058602 therefore proposes the use of a guider catheter having a steerable tip, such that the distal end of the catheter may be steered relative to the rest of the catheter to orientate the distal end of the catheter toward the treatment site.

It has now been observed that the method disclosed in WO2021/058602 has the deficiency that the steerable catheter may cause unacceptable strain and potential further damage at a site that is already in need of repair. The longitudinal axis of a catheter inserted into a blood vessel is substantially the same as the axial direction of the blood vessel lumen. Steering of the steerable tip therefore causes the distal end of the catheter to turn away from the longitudinal axis and toward the wall of the blood vessel. This risks the problem that the tip of the catheter can contact and strain against the wall of the blood vessel at a site which is already weakened due to a cardiovascular defect. Attempting to advance the deployment device from the distal opening of the catheter in this configuration risks further damage to the wall of the blood vessel as well as impeding the proper expansion of the deployment device, potentially resulting in incorrect placement of the patch.

Conventional steerable catheters, in which steerable sections are intended for the purpose of navigating the tip of a catheter through complex anatomical passages are therefore unsuited to applications in which a catheter is used to deploy a treatment device to a sidewall of an anatomical structure. In the context of such applications, simply orienting the catheter tip in the required direction is not sufficient. Instead, the catheter tip must be oriented in the required direction while also creating sufficient free space between the catheter tip and an adjacent anatomical defect to allow for the deployment of a treatment device without unwanted contact of the catheter tip with the defect.

There is therefore a need for improved catheters that facilitate the deployment of medical devices within anatomical lumens, particularly in the case where the treatment site is a wall of an anatomical lumen. Such catheters must be able to orientate the tip of the catheter appropriately for deployment of the medical device within constrained spaces, and without damaging surrounding physiological structures. In particular, there is a need for improved patch deployment systems that are able to deploy cardiovascular repair patches without contacting and/or causing strain at the site of a cardiovascular defect.

### SUMMARY

According to a first aspect, there is provided a steerable catheter comprising a longitudinal tube having a proximal end, a distal end comprising a distal opening, and an internal lumen extending to the distal opening;
wherein a steering zone is provided adjacent to the distal end, wherein the steering zone comprises:
(i) a first steering section; and
(ii) a second steering section, disposed proximally of the first steering section along the longitudinal axis of the catheter;
wherein the first steering section is independently actuatable to steer in a first direction in a first plane, wherein the first plane is parallel to and intersects the longitudinal axis of the catheter;
wherein the second steering section is independently actuatable to steer in a second direction, wherein the second direction is opposite to the first direction; and
wherein the steering zone is configured such that when the second steering section is actuated to its maximum steering angle, the distal end of the catheter cannot intersect a second plane, wherein the second plane is parallel to and intersects the longitudinal axis of the catheter and is perpendicular to the first plane.

According to a second aspect, there is provided a steerable catheter comprising a longitudinal tube having a proximal end, a distal end comprising a distal opening, and an internal lumen extending to the distal opening;
wherein a steering zone is provided adjacent to the distal end, wherein the steering zone comprises:
(i) a first steering section; and
(ii) a second steering section, disposed proximally of the first steering section along the longitudinal axis of the catheter;
wherein the first steering section is independently actuatable to steer in a first direction in a first plane, wherein the first plane is parallel to and intersects the longitudinal axis of the catheter;
wherein the second steering section is independently actuatable to steer in a second direction, wherein the second direction is opposite to the first direction; and
wherein the first steering section has a maximum steering angle of at least 60°.

According to a third aspect, there is provided a steerable catheter comprising a longitudinal tube having a proximal end, a distal end comprising a distal opening, and an internal lumen extending to the distal opening;
wherein a steering zone is provided adjacent to the distal end, wherein the steering zone comprises:
(i) a first steering section, wherein the first steering section is independently actuatable to steer in a first direction in a first plane, wherein the first plane is parallel to and intersects the longitudinal axis of the catheter; and
(ii) a shape-set curve steering section, disposed proximally of the first steering section along the longitudinal axis of the catheter, wherein the shape set curve steering section curves in a second direction and is independently actuatable to steer in the first direction, wherein the second direction is opposite to the first direction; and
wherein the steering zone is configured such that when the shape-set curve steering section is not actuated, the distal end of the catheter cannot intersect a second plane, wherein the second plane is parallel to and intersects the longitudinal axis of the catheter and is perpendicular to the first plane.

According to a fourth aspect, there is provided a steerable catheter comprising a longitudinal tube having a proximal end, a distal end comprising a distal opening, and an internal lumen extending to the distal opening;
wherein a steering zone is provided adjacent to the distal end, wherein the steering zone comprises:
(i) a first steering section, wherein the first steering section is independently actuatable to steer in a first direction in a first plane, wherein the first plane is parallel to and intersects the longitudinal axis of the catheter; and
(ii) a shape-set curve steering section, disposed proximally of the first steering section along the longitudinal axis of the catheter, wherein the shape set curve steering section curves in a second direction and is actuatable to steer in the first direction, wherein the second direction is opposite to the first direction; and
wherein the first steering section has a maximum steering angle of at least 60°.

According to a fifth aspect, there is provided a system for deploying a patch to an anatomical lumen, the system comprising:
(a) a steerable catheter according to the first or second aspect; and
(b) a deployment device, the deployment device comprising:
   (i) a self-expanding deployment structure configured to be in an unexpanded state when positioned and constrained within the lumen of the steerable catheter, and configured to self-expand into an expanded state when displaced through the distal opening and beyond the distal end of the catheter; and
   (ii) a pusher wire that is axially moveable within the lumen of the catheter to move the self-expanding deployment structure between an unexpanded state within the catheter and an expanded state beyond the distal end of the catheter.

According to a sixth aspect, there is provided a system for deploying a patch to an anatomical lumen, the system comprising:
(a) a steerable catheter according to the third or fourth aspects; and
(b) a deployment device, the deployment device comprising:
   (i) a self-expanding deployment structure configured to be in an unexpanded state when positioned and constrained within the lumen of the steerable catheter, and configured to self-expand into an expanded state when displaced through the distal opening and beyond the distal end of the catheter; and
   (ii) a pusher wire that is axially moveable within the lumen of the catheter to move the self-expanding deployment structure between an unexpanded state within the catheter and an expanded state beyond the distal end of the catheter.

According to a seventh aspect, there is provided a method of applying a patch to a site in an anatomical lumen, the method comprising the steps of:
(a) providing a system according to the fifth aspect comprising a catheter according to the first or second aspect, with the self-expanding deployment structure positioned and constrained in an unexpanded state within the lumen of the catheter and further comprising a patch releasably attached to the self-expanding patch deployment structure;
(b) inserting the catheter into the anatomical lumen so as to position the distal opening of the catheter adjacent to the defect site;
(c) actuating the first and second steering sections, to steer the first steering section in the first direction in the first plane and to steer the second steering section in the second direction in the first plane, such that the distal opening of the steerable catheter is oriented toward the defect site;
(d) advancing the pusher wire to move the self-expanding deployment structure into an expanded state beyond the distal opening of the catheter
(e) applying the patch to the defect site;
(f) releasing the patch from the self-expanding deployment structure;
(g) withdrawing the pusher wire to retract the self-expanding deployment structure into the unexpanded state within the lumen of the catheter; and
(h) removing the catheter from the anatomical lumen.

According to an eighth aspect, there is provided a method of applying a patch to a site in an anatomical lumen, the method comprising the steps of:
(a) providing a system according to the sixth aspect comprising a catheter according to the third or fourth aspect, with the self-expanding deployment structure positioned and constrained in an unexpanded state within the lumen of the catheter and further comprising a patch releasably attached to the self-expanding patch deployment structure;
(b) inserting the catheter into the anatomical lumen while actuating the shape-set curve steering section so as to position the distal opening of the catheter adjacent to the defect site;
(c) actuating the first steering section, to steer the first steering section in the first direction in the first plane, and releasing the actuation of the shape-set curve steering section such that the distal opening of the steerable catheter is oriented toward the defect site;
(d) advancing the pusher wire to move the self-expanding deployment structure into an expanded state beyond the distal opening of the catheter
(e) applying the patch to the defect site;
(f) releasing the patch from the self-expanding deployment structure;
(g) withdrawing the pusher wire to retract the self-expanding deployment structure into the unexpanded state within the lumen of the catheter; and
(h) removing the catheter from the anatomical lumen.

According to a ninth aspect, there is provided a method of deploying a medical device using a catheter according to the first or second aspect, the method comprising:
(i) inserting the catheter into an anatomical lumen adjacent to a treatment site;
(ii) actuating the first and second steering sections, to steer the first steering section in the first direction in the first plane and to steer the second steering section in the second direction in the first plane, such that the distal end of the catheter is oriented toward the defect site; and
(iii) deploying the medical device via the distal opening.

According to a tenth aspect, there is provided a method of deploying a medical device using a catheter according to the third or fourth aspect, the method comprising:
(i) inserting the catheter into an anatomical lumen while actuating the shape-set curve steering section so as to position the distal opening of the catheter adjacent to a treatment site;
(ii) actuating the first steering section, to steer the first steering section in the first direction in the first plane, and releasing the actuation of the shape-set curve steering section such that the distal opening of the steerable catheter is oriented toward the defect site; and
(iii) deploying the medical device via the distal opening.

### BRIEF DESCRIPTION OF THE DRAWINGS

FIG. 1A is a schematic view of a prior art steerable catheter having a single steering section.

FIG. 1B is a schematic view of the steerable catheter of FIG. 1A during deployment of a self-expanding deployment structure via the distal end of the catheter.

FIG. 2A is a schematic view of a steerable catheter according to the first aspect in a non-actuated (linear) configuration.

FIG. 2B is a schematic view of the steerable catheter of FIG. 2A in an actuated configuration.

FIG. 3A is a schematic view of the steerable catheter of FIG. 2A showing the arc described by the distal end of the catheter upon actuation of the first steering section.

FIG. 3B shows a close up of the area A of FIG. 3A.

FIG. 4 shows an example of a self-expanding deployment structure 50 in an expanded state beyond the distal end of the steerable catheter.

FIG. 5 depicts part of the descending aorta with a tear in its wall.

FIG. 6A shows a steerable catheter being positioned adjacent to the tear.

FIG. 6B depicts application of a patch to the tear.

FIG. 7 is a schematic view of the system in use to repair a tear in the wall of the ascending aorta.

FIG. 8 is a further schematic view of a patch deployment system including a handle structure.

FIGS. 9A to 9F show the steerable catheter in various actuated configurations.

FIGS. 10A to 10D are schematic cross-sectional views of the proximal shaft section, second (proximal) steering section, connecting section and first (distal) steering section of a catheter according to the present disclosure.

### DETAILED DESCRIPTION

In the following description, the catheters of the present disclosure are described in detail with particular reference to the catheters of the first and second aspects of the present disclosure. It will be understood that, to the extent that any features described herein are common to more than one aspect of the present disclosure, that disclosure is applicable to all aspects of the present disclosure.

The steerable catheters of the first and second aspects of present disclosure are generally defined by a distal end and a proximal end. The distal end comprises a distal opening and an internal lumen extends through the catheter to the distal opening. A steering zone is provided adjacent to the distal end, wherein the steering zone comprises a first steering section and a second steering section disposed proximally of the first steering section along the longitudinal axis of the catheter. The catheters generally further comprise a substantially straight shaft section extending from the proximal end to the second steering section and optionally a tip section extending distally from the first steering section to the distal end.

The first and second steering sections are independently actuatable to steer in opposite directions. It has been found that steerable catheters according to the present disclosure can overcome the deficiencies of steerable catheters that have only a single steering section as set out above.

Actuation of the first steering section has the effect of orienting the distal end of the catheter toward a side wall of the vasculature, at a site where treatment is necessary. While this achieves the correct orientation of the distal opening for the deployment of an appropriate medical device, it also results in displacement of the distal end from the longitudinal axis of the catheter in the direction of the treatment site (referred to herein as the “forward direction”). As set out above, this displacement can result in the distal end of the catheter contacting and straining against the wall of the anatomical lumen at the treatment site and/or leaving inadequate space between the distal opening of the catheter and the treatment site for the deployment of an appropriate medical device.

The provision of the second steering section can address this problem by enabling a displacement of the distal end of the catheter in the direction away from the treatment site (referred to herein as the “rearward direction”), thereby compensating for the displacement of the distal end of the forward direction due to actuation of the first steering section. This allows for the distal opening of the catheter to be oriented toward a treatment site while creating free space between the distal opening and the treatment site to allow the deployment of an appropriate treatment device.

The longitudinal axis of the catheter is defined herein as the central axis of the internal lumen as determined immediately proximal of the steering zone. Accordingly, the distal end of the catheter will be in line with the longitudinal axis when both steering sections are positioned at a steering angle of 0°. The steering angle of the second steering section is always determined relative to the longitudinal axis of the catheter.

The operation of the catheter can be described by reference to two perpendicular planes that each lie parallel to and intersect the longitudinal axis of the catheter. The first plane is defined as the steering plane of the first steering section. The second plane is parallel to and intersects the longitudinal axis of the catheter and is perpendicular to the first plane.

The first and second planes may be defined by reference to Cartesian coordinates where the longitudinal axis of the catheter is defined as the y-axis. The steering plane of the first steering section is defined as the x,y plane. Steering of the first steering section in the first direction in the first plane results in displacement of the distal end of the catheter in the forward direction (defined herein as the x+ direction in the x,y plane). Steering of the second steering section in the second direction causes a compensatory displacement of the distal end of the catheter in the rearward direction (i.e. the x− direction in the x,y plane). When both the first and second steering sections are actuated, the steering zone has a generally S-shaped configuration.

Using the same Cartesian coordinates, the second plane is the y,z plane. In accordance with the first aspect of the present disclosure, the catheter is configured such that when the second steering section is positioned at its maximum steering angle, the distal end of the catheter cannot intersect the second plane.

Accordingly, the first and second steering sections cooperate such that any movement of the distal end of the catheter in the x+ direction upon actuation of the first steering section is fully compensated when the second steering section is actuated to its maximum steering angle, such that there is no net forward movement of the distal end of the catheter from the second plane towards a treatment site.

Put in other words, the catheter is configured such that when the second steering section is actuated to its maximum steering angle, the arc described by the distal end of the catheter upon actuation of the first steering section to its maximum steering angle does not intersect the second plane. This creates free space between the distal end of the catheter and a treatment site located in the forward direction which allows for deployment of a medical device from the distal opening of the catheter without causing strain at the treatment site.

Whereas a catheter of the type described in WO2021/058602, having only a single steering section, necessarily results in displacement of the catheter tip from the longitudinal axis toward the treatment site, the catheters of the present disclosure provide a steering zone in which the displacement of the distal end from the longitudinal axis toward the treatment site is compensated by the second steering section.

The maximum steering angle of the first steering section defines the capacity of the catheter to orient the distal opening toward a treatment site. In accordance with the second aspect of the present disclosure the first steering section has a maximum steering angle of at least 60°.

As used herein, the maximum steering angle of each steering section defines the maximum steering range of the steering section. For instance, a steering section with a maximum steering angle of 60° will be steerable through a range of angles, typically from 0° up to a maximum of 60° in the respective steering direction. The range of angles that can be adopted by each steering section is referred to herein as the “steering range”.

The steering angle that is required depends on the shape of the anatomical lumen at the treatment site. For example, in the case that the treatment site lies essentially parallel to the longitudinal axis of the catheter, then a steering angle of greater than 90° would usually be appropriate for the first steering section to enable orientation of the distal opening towards the treatment site while taking into account the opposite curvature of the second steering section. For example, a steering angle of 110° of the first steering section will orient the catheter tip perpendicularly to the longitudinal axis of the catheter when the second steering section adopts a steering angle of 20°. For curved areas of the vasculature, a lower steering angle may be sufficient to position the catheter tip perpendicularly to the anatomical wall.

A preferred steerable catheter incorporates the features of both the catheters of both the first and second aspects of the present disclosure. Accordingly, a preferred steerable catheter according to the present disclosure comprises a longitudinal tube having a proximal end, a distal end comprising a distal opening and an internal lumen extending to the distal opening;
wherein a steering zone is provided adjacent to the distal end, wherein the steering zone comprises:
(i) a first steering section; and
(ii) a second steering section, disposed proximally of the first steering section along the longitudinal axis of the catheter;
wherein the first steering section is independently actuatable to steer in a first direction in a first plane, wherein the first plane is parallel to and intersects the longitudinal axis of the catheter;
wherein the second steering section is independently actuatable to steer in a second direction in the first plane, wherein the second direction is opposite to the first direction;
wherein the steering zone is configured such that when the second steering section is actuated to its maximum steering angle, the distal end cannot intersect a second plane, wherein the second plane is parallel to and intersects the longitudinal axis of the catheter and is perpendicular to the first plane; and
wherein the first steering section has a maximum steering angle of at least 60°.

A steerable catheter according to the first and second aspects of the present disclosure is preferably configured such that when the second steering section is actuated to its maximum steering angle, the distal end of the catheter is separated from the second plane by a minimum distance throughout the steering range of the first steering section. Optionally, the minimum distance is at least 0.1 mm, or at least 0.5 mm, or at least 1 mm, or at least 2 mm, or at least 5 mm, or at least 10 mm, or at least 15 mm, or at least 20 mm, or at least 25 mm, or at least 30 mm.

Put in other words, the steerable catheters of the present disclosure are preferably configured such that when the second steering section is actuated to its maximum steering angle, the minimum distance between the arc described by the distal end of the catheter upon actuation of the first steering section to its maximum steering angle and the second plane is at least 0.1 mm, or at least 0.5 mm, or at least 1 mm, or at least 2 mm, or at least 5 mm, or at least 10 mm, or at least 15 mm, or at least 20 mm, or at least 25 mm, or at least 30 mm.

In general, the greater the separation of the distal end of the catheter from the second plane, the more free space is made available for the deployment of a medical device via the distal opening of the catheter.

The first steering section preferably has a maximum steering angle (in the first direction) of at least 60°, or at least 70°, or at least 80°, or at least 90°. More preferably, the first steering section has a maximum steering angle that is greater than 90°, for example, at least 100°, or at least 110°, or at least 120°, or at least 130°, or at least 140°, or at least 150°, or at least 160°. Optionally, the maximum steering angle of the first steering section is no more than 270°, or no more than 240°, or no more than 210°, or no more than 180°, or no more than 170°.

The first steering section preferably has a steering range (in the first direction) from 0° to at least 60°, or from 0° to at least 70°, or from 0° to at least 80°, or from 0° to at least 90°. More preferably, the first steering section has a steering range from 0° to greater than 90°, for example, from 0° to at least 100°, or from 0° to at least 110°, or from 0° to at least 120°, or from 0° to at least 130°, or from 0° to at least 140°, or from 0° to at least 150°, or from 0° to at least 160°.

Optionally, the first steering section has a steering range (in the first direction) from 0° to no more than 270°, or from 0° to no more than 240°, or from 0° to no more than 210°, or from 0° to no more than 180°, or from 0° to no more than 170°.

The first steering section preferably has a steering radius at its maximum steering angle in the range from 5 to 200 mm, or from 5 to 100 mm, or from 8 to 50 mm, or from 10 to 40 mm, or from 12 to 30 mm, or from 15 to 25 mm. The steering radius of the first steering section as defined herein is measured with reference to the centreline of the first steering section.

The second steering section preferably has a maximum steering angle (in the second direction) of at least 10°, or at least 20°, or at least 30°, or at least 40°, or at least 50°, or at least 60°, or at least 70°, or at least 80°, or at least 90°. Optionally, the maximum steering angle of the second steering section is no more than 210°, no more than 180°, or no more than 150°, or no more than 120°, or no more than 110°, or no more than 100°.

The second steering section preferably has a steering range from 0° to at least 10°, or from 0° to at least 20°, or from 0° to at least 30°, or from 0° to at least 40°, or from 0° to at least 50°, or from 0° to at least 60°, or from 0° to at least 70°, or from 0° to at least 80°, or from 0° to at least 90°.

Optionally, the second steering section has a steering range (in the second direction) from 0° to no more than 210°, or from 0° to no more than 180°, or from 0° to no more than 150°, or from 0° to no more than 120°, or from 0° to no more than 110°, or from 0° to no more than 100°, or from 0° to no more than 90°, or from 0° to no more than 80°, or from 0° to no more than 70°, or from 0° to no more than 60°, or from 0° to no more than 50°.

The second steering section preferably has a steering radius at its maximum steering angle in the range from 10 to 700 mm, or from 10 to 500 mm, or from 15 to 200 mm, or from 25 to 150 mm, or from 30 to 120 mm, or from 35 to 100 mm, or from 40 to 80 mm, or from 40 to 60 mm, or from 40 to 50 mm. The steering radius of the second steering section as defined herein is measured with reference to the centreline of the first steering section.

It is not excluded that the second steering section can steer in a different plane to the first steering section (i.e. such that the displacement includes a component in the z-direction using the Cartesian coordinate system described above). However, the angle of the second steering section is defined herein in the second direction (the x− direction) and therefore excludes any component in the z-direction. In other words, the angle of the second steering section is defined in plan view as determined by looking along the z-direction. Preferably, the first and second steering sections steer in substantially the same plane (e.g. the steering planes differ by no more than ±30°, preferably no more than ±20°, more preferably no more than ±10°, more preferably no more than ±5°, as viewed along the y-axis). Most preferably, the first and second steering sections steer in the same plane. For the avoidance of doubt, the first plane (the x,y plane) is defined herein when the steering angle of the second steering section is 0°

Preferably, the first and second steering sections are independently actuatable to orientate the distal end of the steerable catheter such that it is perpendicular to the longitudinal axis of the catheter. It will be understood that the distal end of the steerable catheter will be oriented perpendicular to the longitudinal axis when the difference between the steering angle of the first steering section in the first direction, and the steering angle of the second steering section in the second direction is 90°. Accordingly, it is preferred that the first steering section has a maximum steering angle of at least (90+n)° and the second steering section has maximum steering angle of at least n°, for example where n is at least 10, or at least 20, or at least 30, or at least 40, or at least 50.

Preferably, the maximum steering angle of the first steering section is at least 100°, or at least 110°, or at least 120°, or at least 130°, or at least 140°, or at least 150°, or at least 160° and the maximum steering angle of the second steering section is at least 10°.

More preferably, the maximum steering angle of the first steering section is at least 100°, or at least 110°, or at least 120°, or at least 130°, or at least 140°, or at least 150°, or at least 160° and the maximum steering angle of the second steering section is at least 30°.

More preferably, the maximum steering angle of the first steering section is at least 100°, or at least 110°, or at least 120°, or at least 130°, or at least 140°, or at least 150°, or at least 160° and the maximum steering angle of the second steering section is at least 50°.

More preferably, the maximum steering angle of the first steering section is at least 100°, or at least 110°, or at least 120°, or at least 130°, or at least 140°, or at least 150°, or at least 160° and the maximum steering angle of the second steering section is at least 70°.

More preferably, the first and second steering sections are independently actuatable to orientate the distal end of the steerable catheter such that it is perpendicular to the longitudinal axis of the catheter, and wherein the minimum distance between the distal end of the catheter and the second plane is at least 0.1 mm, or at least 0.5 mm, or at least 1 mm, or at least 2 mm, or at least 5 mm, or at least 10 mm, or at least 15 mm, or at least 20 mm, or at least 25 mm, or at least 30 mm when the distal end is in said perpendicular orientation and when the second steering section is simultaneously actuated to its maximum steering angle.

In particular, the steerable catheters of the present disclosure are preferably configured such that the first and second steering sections are independently actuatable to orientate the distal end of the steerable catheter such that it is perpendicular to the longitudinal axis of the catheter when the second steering section is actuated to its maximum steering angle. Preferably, the minimum distance between distal end of the catheter and the second plane in said configuration is at least 0.1 mm, or at least 0.5 mm, or at least 1 mm, or at least 2 mm, or at least 5 mm, or at least 10 mm, or at least 15 mm, or at least 20 mm, or at least 25 mm, or at least 30 mm.

The dimensions of the steering zone are selected such that the steerable catheter is actuatable within the confines of an anatomical lumen requiring treatment. In general, for cardiovascular, use, the first steering section preferably has a length in the range from 5 to 180 mm, or from 10 to 180 mm, or from 20 to 150 mm, or from 30 to 120 mm, or from 40 to 100 mm, or from 45 to 90 mm, or from 50 to 80 mm. The length of the first steering section is defined as the perimetral length of the first steering section at its maximum steering angle.

The second steering section preferably has a length in the range from 5 to 180 mm, or from 10 to 180 mm, or from 20 to 150 mm, or from 30 to 120 mm, or from 40 to 100 mm, or from 45 to 90 mm, or from 50 to 80 mm. The length of the second steering section is defined as the perimetral length of the second steering section at its maximum steering angle.

Still more preferably, both the first and second steering sections have a length in the range from 5 to 180 mm, or from 10 to 180 mm, or from 20 to 150 mm, or from 30 to 120 mm, or from 40 to 100 mm, or from 45 to 90 mm, or from 50 to 80 mm, wherein the respective lengths are defined as the perimetral length of each steering section at its maximum steering angle.

Optionally, the first steering section has a length L1 and the second steering section has a length L2, wherein L1>L2.

Preferably, the sum of L1+L2 is in the range from 10 to 200 mm, or from 20 to 200 mm, or from 25 to 150 mm, or from 30 to 120 mm.

The steering zone optionally further comprises a substantially straight connecting section disposed between the first steering section and the second steering section. The effect of the straight connecting section is essentially to increase the displacement of the distal end of the catheter in the rearward direction approximately in proportion to its length. The connecting section may have a length L3, wherein L3 is in the range from 5 to 100 mm, or from 10 to 90 mm, or from 15 to 80 mm, or from 20 to 70 mm, or from 25 to 60 mm, or from 30 to 50 mm.

Preferably, the steering zone has a total length (L1+L2+L3) in the range from 20 to 300 mm, or from 50 to 300 mm, or from 55 to 250 mm, or from 60 to 200 mm, or from 65 to 180 mm, or from 70 to 160 mm, or from 75 to 150 mm.

Each steering section may be independently steerable in one or two directions. In the case that each steering section is steerable in one direction, then the respective steering directions must be in opposite directions in the first plane, as set out above. However, it is not excluded that one or both steering sections may be steerable in both directions. It will be understood that only a configuration in which the first and second steering sections are steered in opposite directions is suitable for deployment of a medical device while taking advantage of the ability of the catheter of the present disclosure to create free space. However, the ability to steer both steering sections in the same direction may be of utility during navigation of the steerable catheter to a treatment site.

In one configuration, the first and second steering sections are each steerable in a single, respectively opposite, direction.

In another configuration, the first steering section is steerable in both directions and the second steering section is steerable in a single direction.

In another configuration, the first steering section is steerable in a single direction and the second steering section is steerable in both directions.

In another configuration, the first and second steering sections are each steerable in both directions.

Unless specified otherwise, references herein to the steering angles of the first and second steering sections refer to the steering angle of the first steering section in the first steering direction, and to the steering angle of the second steering section in the second (i.e. opposite) direction.

In the case that the first steering section can steer in both directions, the maximum steering angle in the second steering direction may be the same as, or different from, the maximum steering angle in the first steering direction as defined above. Likewise, in the case that the second steering section can steer in both directions, the maximum steering angle in the first steering direction may be the same as, or different from, the maximum steering angle in the second steering direction as defined above.

As set out above, the catheter of the present disclosure generally comprises a proximal shaft section extending from the proximal end to the steering zone. The purpose of the proximal shaft section is primarily to span the distance between an insertion site, such as an incision, where the catheter is inserted into an anatomical lumen and the treatment site. The shaft section is generally substantially straight although it is not excluded that it may have one or more pre-set curves. The shaft section may be resiliently flexible so that it may conform to the local shape of an anatomical lumen between the insertion site and the treatment site.

The steerable catheter may optionally further comprise a tip section extending distally from the first steering section to the distal end. The tip section is essentially a non-steerable terminal portion of catheter tube. Since the tip section extends beyond the first steering section, it increases the proximity of the distal end to the second plane and/or the treatment site. In other words, it reduces the free space between the distal opening and the treatment site. Accordingly, the length of the tip section, defined herein as L4, is preferably no more than 50 mm, or no more than 40 mm, or no more than 30 mm, or no more than 20 mm, or no more than 10 mm, or no more than 8 mm, or no more than 6 mm, or no more than 5 mm, or no more than 4 mm, or no more than 2 mm. Nonetheless, a linear tip section of non-negligible length may facilitate the deployment of a medical device from the distal opening, since the medical device is not deflected within the curvature of the first steering section immediately prior to deployment.

The outer diameter of the steering zone in French gauge (Fr) is preferably no more than 40 Fr, or no more than 30 Fr, or no more than 25 Fr, or no more than 20 Fr, or no more than 18 Fr, or no more than 16 Fr, or no more than 14 Fr, or no more than 12 Fr, or no more than 10 Fr, or no more than 8 Fr, or no more than 7 Fr, or no more than 6 Fr, or no more than 5 Fr. Preferably, the outer diameter of the catheter is substantially uniform from the proximal end to the distal end, including the steering zone, and is no more than 40 Fr, or no more than 30 Fr, or no more than 25 Fr, or no more than 20 Fr, or no more than 18 Fr, or no more than 16 Fr, or no more than 14 Fr, or no more than 12 Fr, or no more than 10 Fr, or no more than 8 Fr, or no more than 7 Fr, or no more than 6 Fr, or no more than 5 Fr. One unit of French gauge is equivalent to ⅓ mm. Therefore, 18 Fr is equivalent to 6 mm.

Preferably, internal lumen of the steerable catheter has a circular cross-section.

As used herein, the term “independently actuatable” means that the first and second steering sections are actively steerable, independently from each other, upon application of an actuation mechanism. Whereas some known catheters comprise resilient zones that are passively deflectable, passive deflection is not capable of creating free space since it relies on contact between a catheter and the side walls of an anatomical lumen.

Preferably, actuation of the steering sections is by first and second pull wires that are configured to actuate each of the first and second steering sections, respectively. Suitably, each pull wire is disposed within one or more guide lumens that extend from the proximal end of the catheter to the steering zone.

The catheter optionally comprises a handle portion at the proximal end, the handle portion comprising first and second actuators that are respectively configured for manual actuation of each of the first and second steering sections. Each actuator may be configured as a rotatable annular band around the handle. Each actuator may be lockable so as to fix the steering angle even when the actuator is released.

Alternatively, the proximal end of the catheter may comprise electronic actuators, optionally with an interface for a computer to facilitate computerised actuation of the first and second steering sections. The proximal end of the catheter may further comprise means for connecting the catheter to a robotic arm.

In the case that one or both of the first and second steering sections is steerable in two directions, additional pull wires may be provided to actuate steering in both directions.

The catheter of the present disclosure can be manufactured from various materials and combinations of materials, such as stainless steel, nitinol, and polymers of different durometers and lengths (e.g. a PTFE extrusion). The catheter structure may comprise multiple layers, such as a coiled pipe, metallic or polymeric wires, a braided structure, polymer extrusions forming distinct lumens, etc. The layers within the catheter body can assume a continuous or discontinuous form or section along the entire shaft, tailored to achieve the desired flexibility for each section. The catheter optionally further comprises an outer coating, such as a polymeric coating, which provides a smooth catheter surface that can reduce friction and trauma on insertion. The catheter optionally further comprises an internal liner, such as a polymeric liner, more preferably a fluoropolymer liner, which forms the wall of the internal lumen and can provide a smooth internal surface with a low coefficient of friction.

The first and second steering sections may each have directional anisotropy, such that the resistance of each steering section to bending in the steering plane is lower than the resistance to bending in a direction out of the steering plane. In other words, there may be limited directional degrees of freedom to the flexibility of each steering section, such that each steering section is relatively flexible in the intended steering direction and relatively inflexible in other directions. As a result, the deflection of each steering section is substantially constrained to the intended steering direction.

In conventional steerable catheters, in which the purpose of the steerability is primarily to facilitate the navigation of the catheter tip through complex anatomical structures, it is advantageous for a steering zone to have multiple degrees of steering freedom in order that the catheter tip can be manipulated as necessary to achieve the required navigation. However, the catheters of the present disclosure address a different function, namely the need to create free space between a catheter tip and the sidewall of an anatomical structure. The creation of free space requires the actuation of two steering sections with control over both the relative direction of steering of each steering section and the steering angle of each steering section.

In the case that the steering sections would have multiple degrees of freedom, the simultaneous manipulation of both steering sections in such a way as to create the required free space in the required direction is complex to achieve, since it requires simultaneous control of each steering angle and each steering direction. In addition, the possibility of passive deflections of the steering zone by anatomical structures presents another potential difficulty in achieving the relative positioning of both steering sections that is required to achieve free space.

By limiting the directional degrees of freedom of each steering section, the present disclosure provides a steerable catheter which is optimised for the purpose of creating free space and which is simpler both to operate and manufacture.

Accordingly, the first steering section preferably has lower resistance to bending in the first plane (x,y-plane) than in the second plane (y,z-plane). Preferably, the bending resistance in the first plane is no more than 50%, or no more than 20%, or no more than 10%, or no more than 5% of the bending resistance in the second plane. More preferably, the first steering section is non-flexible in the second plane (y,z-plane, in both the z− and z+ directions).

Optionally, the first steering section is flexible in both directions in the first plane (x+ direction and x− direction) such that the first steering section is steerable throughout the first plane, while being relatively inflexible, or even non-flexible, in the second plane (y,z-plane, in both the z− and z+ directions). In this case, the first steering section has two directional degrees of freedom (namely the forward (x+) direction and the rearward (x−) direction).

Alternatively, the first steering section may also have lower resistance to bending in the forward direction (x+ direction) than in both the rearward direction (x− direction) and the z-direction such that the first steering section is flexible in the forward direction (x+ direction) in the first plane, while being relatively inflexible, or even non-flexible, in both the rearward direction (x− direction) and the second plane (y,z-plane, in both the z− and z+ directions). In this case, the first steering section has one directional degree of freedom (namely the forward (x+) direction).

Similarly, the second steering section preferably has lower resistance to bending in the steering plane than in the direction perpendicular to the steering plane.

As noted above, the steering plane of the second steering section is preferably the same as the steering plane of the first steering section (i.e. the x,y-plane), but it is not excluded that the steering planes of the first and second steering sections may be offset such that the steering plane of the second steering section includes a component in the z-direction. In the following discussion, the steering of the second steering section is described by reference to the forward and rearward directions, which correspond generally to the x+ and x− directions respectively, but optionally include a component in the z-direction.

Preferably, the bending resistance of the second steering section in the steering plane is no more than 50%, or no more than 20%, or no more than 10%, or no more than 5% of the bending resistance in the direction perpendicular to the steering plane. More preferably, the second steering section is non-flexible in the direction perpendicular to the steering plane.

Optionally, the second steering section is flexible in both directions in the steering plane (forward direction and rearward direction) such that the second steering section is steerable throughout the steering plane, while being relatively inflexible, or even non-flexible, in the direction perpendicular to the steering plane. In this case, the second steering section has two directional degrees of freedom (forward and rearward in the steering plane).

Alternatively, the second steering section may also have lower resistance to bending in the rearward direction than in both the forward direction and the direction perpendicular to the steering plane such that the first steering section is flexible in the rearward direction in the steering plane, while being relatively inflexible, or even non-flexible, in the both the forward direction and the direction perpendicular to the steering plane. In this case, the second steering section has one directional degree of freedom (rearward in the steering plane).

In the case that the first and second steering sections have directional anisotropy, the steering planes of the first and second steering sections preferably have a fixed relative orientation. In other words, the first and second steering sections are preferably connected (optionally via a connecting section), such that the steering planes cannot rotate relative to one another. Preferably, the first and second steering sections have a fixed relative orientation in which both steering planes are the first plane (x,y-plane).

Preferably, the first and second steering sections each comprise an articulated hypotube section. As used herein, an articulated hypotube section refers to a catheter hypotube section comprising a plurality of flexibility enhancing features that impart the tube section with increased flexibility in the intended steering plane relative to the flexibility out of the steering plane. Preferably, the flexibility enhancing features comprise a plurality of cuts in the wall of the hypotube, wherein the geometric arrangement, or pattern, of the plurality of cuts is selected both to increase the flexibility of the catheter in the intended steering direction and to define the steering range (maximum steering angle) of the steering section.

More preferably, the steering zone (comprising the first and second steering sections and the optional connecting section disposed between the first and second steering sections) is integrally formed from a single hypotube comprising first and second articulated zones corresponding to each of the first and second steering sections. Optionally, a non-articulated zone—corresponding to the above-mentioned connecting section—is provided between the first and second articulated zones. Preferably, each of the first and second articulated zones comprises a plurality of cuts in the wall of the hypotube, wherein the pattern of cuts in each of the first and second articulated zones is selected to increase the flexibility of each steering section in the respective steering directions.

The hypotube may be used in combination with other structural components that provide additional functionality to the steering zone. For instance, the hypotube may be surrounded by an outer jacket comprising one or more layers, such as a coiled pipe, metallic or polymeric wires, or a braided structure. The outer jacket may further comprise a polymeric coating, which optionally extends to the entire length of the catheter. The hypotube may also be provided with an internal liner, such as a polymeric liner, more preferably a fluoropolymer liner, that forms the wall of the catheter lumen in the steering zone. The internal liner optionally extends to the entire length of the catheter internal lumen. An internal liner may be useful to reduce the coefficient of friction of the internal wall of the catheter.

A further advantage of using articulated steering zones in the catheter of the present disclosure is that the flexibility of the steering zones is increased substantially relative the flexibility of the proximal shaft section. In prior steerable catheters, the force applied to a pull wire to actuate a steering zone can act not only on the steering zone but also on the proximal portion of the catheter shaft, resulting in an unwanted deformation of the catheter shaft in addition to the desired steering action at the distal portion of the catheter. Deformation of the catheter shaft is undesirable since it reduces control of the positioning of the catheter tip and can result in stress to anatomical structures between the catheter insertion point and the treatment site. It has therefore been found that the catheters of the present disclosure allow for actuation of both sections of the steering zone without substantial deformation of the proximal shaft section of the catheter, thereby obviating the need for the use of additional measures (e.g. stiffening members) to reduce deformation of the proximal shaft section.

In the absence of the need to provide additional measures, such as stiffening members, to reduce the deformation of the proximal shaft section, the catheters of the present disclosure can have a high ratio of the internal lumen diameter to the external diameter in the proximal shaft section. Preferably, the ratio of the internal lumen diameter to the external diameter of the catheter in the proximal shaft section is at least 0.60, or at least 0.65, or at least 0.70, or at least 0.75, or at least 0.80, or at least 0.85, or at least 0.90.

More preferably, the ratio of the internal lumen diameter to the external diameter of the catheter is at least 0.60, or at least 0.65, or at least 0.70, or at least 0.75, or at least 0.80, or at least 0.85, or at least 0.90 throughout the proximal shaft section and the steering zone.

The catheter of the present disclosure may optionally further comprise a carrier catheter extending through internal lumen of the steerable catheter, wherein the carrier catheter is axially movable with respect to the steerable catheter. The carrier catheter comprises an internal lumen and is preferably passively flexible in response to actuation of the steerable catheter.

The third and fourth aspects of the present disclosure correspond to the first and second aspects in all respects, except that the second steering section is substituted by a shape-set curve steering section. As used herein, a shape-set curve steering section refers to a steering section of the catheter that adopts a preset curvature in the absence of any actuation force and which is actuatable to steer in the direction opposite to the direction of the preset curvature. The second steering section and the shape-set curve steering section therefore perform the same function except that effect of actuation is opposite. The first steering section requires actuation to increase the angle of curvature in the second direction whereas the shape-set curve steering section requires actuation to reduce the angle of curvature in the second direction.

These two catheter designs are therefore mutually complementary and allow for the curvature of the proximal steering section in the deployment configuration to be achieved either actively or passively.

Accordingly, all of the features disclosed above with respect to catheters of the first and second aspects of the present disclosure are applicable to an equivalent catheter in which the second steering section is substituted by a shape-set curve steering section that curves in the second direction and is independently actuatable to steer in the first direction. This applies in particular, but not exclusively, to the features of the first steering section (including the dimensions and maximum steering angle thereof), the substantially straight connecting section, the tip section, the shaft section, the tip section, and the proximal portion (including handle, actuators and/or electronic interfaces).

A preferred catheter incorporates the features of both the third and fourth catheters. Accordingly, a preferred steerable catheter comprises a longitudinal tube having a proximal end, a distal end comprising a distal opening, and an internal lumen extending to the distal opening;
wherein a steering zone is provided adjacent to the distal end, wherein the steering zone comprises
(i) a first steering section, wherein the first steering section is independently actuatable to steer in a first direction in a first plane, wherein the first plane is parallel to and intersects the longitudinal axis of the catheter; and
(ii) a shape-set curve steering section, disposed proximally of the first steering section along the longitudinal axis of the catheter, wherein the shape set curve steering section curves in a second direction and is independently actuatable to steer in the first direction, wherein the second direction is opposite to the first direction;
wherein the steering zone is configured such that, when the shape-set curve steering section is not actuated, the distal end of the catheter cannot intersect a second plane, wherein the second plane is parallel to and intersects the longitudinal axis of the catheter and is perpendicular to the first plane; and
wherein the first steering section has a maximum steering angle of at least 60°.

A catheter according to the third aspect of the present disclosure is preferably configured such that the distal end of the catheter is separated from the second plane by a minimum distance throughout the steering range of the first steering section. Optionally, the minimum distance is at least 0.1 mm, or at least 0.5 mm, or at least 1 mm, or at least 2 mm, or at least 5 mm, or at least 10 mm, or at least 15 mm, or at least 20 mm, or at least 25 mm, or at least 30 mm.

Put in other words, the catheter of the first aspect of the present disclosure is configured such that the minimum distance between the arc described by the distal end of the catheter upon actuation of the first steering section to its maximum steering angle and the second plane is at least 0.1 mm, or at least 0.5 mm, or at least 1 mm, or at least 2 mm, or at least 5 mm, or at least 10 mm, or at least 15 mm, or at least 20 mm, or at least 25 mm, or at least 30 mm.

Preferably, the first steering section is independently actuatable to orientate the distal end of the steerable catheter such that it is substantially perpendicular to the longitudinal axis of the catheter. Accordingly, it is preferred that the first steering section has a maximum steering angle of at least (90+m)° where m defines the angle of the shape-set curve, for example where m is at least 10, or at least 20, or at least 30, or at least 40, or at least 50.

Preferably, the angle of the shape-set curve steering section in the absence of actuation is in the range from 10° to 210°, or from 10° to 180°, or from 10° to 150°, or from 10° to 120°, or from 10° to 90°, or from 15° to 60°, or from 20° to 45°. As set out above, the angle of the shape-set curve steering section is defined herein without any component in the z-direction (i.e. in plan view looking along the z-direction.

The shape-set curve steering section preferably has a steering radius at its maximum bending angle in the range from 10 to 700 mm, or from 10 to 500 mm, or from 15 to 200 mm, or from 25 to 150 mm, or from 30 to 120 mm, or from 35 to 100 mm. The steering radius of the shape-set curve steering section as defined herein is measured with reference to the centreline of the shape-set curve steering section.

The shape-set curve steering section preferably has a length in the range from 5 to 180 mm, or from 10 to 180 mm, or from 20 to 150 mm, or from 30 to 120 mm, or from 40 to 100 mm, or from 45 to 90 mm, or from 50 to 80 mm. The length of the shape-set curve steering section is defined as the perimetral length of the shape-set curve steering section at its shape-set angle.

Optionally, both the first steering section and the shape-set curve steering section have a length in the range from 5 to 180 mm, or from 10 to 180 mm, or from 20 to 150 mm, or from 30 to 120 mm, or from 40 to 100 mm, or from 45 to 90 mm, or from 50 to 80 mm, wherein the respective lengths are defined as the respective perimetral lengths as defined above.

Optionally, the first steering section has a length L1 and the shape-set curve steering section has a length L5, wherein L1>L5.

Preferably, the steering zone has a total length (L1+L3+L5) in the range from 20 to 300 mm, or from 50 to 300 mm, or from 55 to 250 mm, or from 60 to 200 mm, or from 65 to 180 mm, or from 70 to 160 mm, or from 75 to 150 mm.

Preferably, actuation of the first steering section is by a first pull wire. Suitably, the first pull wire is disposed within a guide lumen that extends from the proximal end of the catheter to the steering zone.

In accordance with the third and fourth aspects, the first steering section may be steerable in one or two directions. In the case that the first steering section is steerable in one direction, then the steering direction must be in the opposite direction to the curvature of the shape-set curve, as set out above. As above, a configuration in which the first steering section is steered in the opposite direction to the shape-set curve is suitable for deployment of a medical device while taking advantage of the ability of the catheter of the present disclosure to create free space. However, the ability to steer the first steering sections in the same direction as the shape-set curve may be of utility during navigation of the steerable catheter to a treatment site.

In accordance with the third and fourth aspects, the first steering section may have directional anisotropy, such that the resistance of the first steering section to bending in the steering plane is lower than the resistance to bending in a direction out of the steering plane.

Accordingly, the first steering section preferably has lower resistance to bending in the first plane (x,y-plane) than in the second plane (y,z-plane). Preferably, the bending resistance in the first plane is no more than 50%, or no more than 20%, or no more than 10%, or no more than 5% of the bending resistance in the second plane. More preferably, the first steering section is non-flexible in the second plane (y,z-plane, in both the z− and z+ directions).

Optionally, the first steering section is flexible in both directions in the first plane (x+ direction and x− direction) such that the first steering section is steerable throughout the first plane, while being relatively inflexible, or even non-flexible, in the second plane (y,z-plane, in both the z− and z+ directions). In this case, the first steering section has two directional degrees of freedom (namely the forward (x+) direction and the rearward (x−) direction).

Alternatively, the first steering section may also have lower resistance to bending in the forward direction (x+direction) than in both the rearward direction (x− direction) and the z-direction such that the first steering section is flexible in the forward direction (x+ direction) in the first plane, while being relatively inflexible, or even non-flexible, in both the rearward direction (x− direction) and the second plane (y,z-plane, in both the z− and z+ directions). In this case, the first steering section has one directional degree of freedom (namely the forward (x+) direction).

In the case that the first steering section has directional anisotropy, the steering plane of the first steering section preferably has a fixed relative orientation to the steering plane of the shape-set curve steering section. Preferably, the steering planes of the first steering section and the shape-set curve steering section both lie in the first plane (x,y-plane).

Preferably, the shape-set curve steering section also has lower resistance to bending in the steering plane than in the direction perpendicular to the steering plane. Preferably, the bending resistance of the shape-set curve steering section in the steering plane is no more than 50%, or no more than 20%, or no more than 10%, or no more than 5% of the bending resistance in the direction perpendicular to the steering plane. More preferably, the shape-set curve steering section is non-flexible in the direction perpendicular to the steering plane.

The fifth and sixth aspects of the present disclosure provide systems for deploying a patch to an anatomical lumen comprising a catheter according to any of the first to fourth aspects of the present disclosure and a self-expanding deployment structure as defined above.

The self-expanding deployment structure is configured to deploy a patch to a defect in an anatomical lumen. The self-expanding deployment structure is configured to hold the patch in a folded configuration while in an unexpanded state and to unfold the patch for deployment after self-expanding into the expanded state. Preferably, the self-expanding deployment structure in the expanded state supports the unfolded patch in a plane that is substantially perpendicular to the end of the catheter. In this manner, the self-expanding deployment structure forms a support that is substantially perpendicular to the longitudinal axis of the pusher wire which can then be used to press a patch onto a patch application site.

An example of a suitable self-expanding deployment structure for use in the system of the present disclosure is disclosed in WO2021/058602. The deployment device of WO2021/058602 comprises a pusher wire having a proximal end and a distal end; and a plurality of deployment wires wherein each deployment wire has a first end and a second end, wherein the first end and the second end are connected to the distal end of the pusher wire. Each of the deployment wires is configured to be in an unexpanded state when positioned and constrained within a catheter and to self-expand into an expanded state when positioned beyond a distal end of the catheter. In the expanded state, at least a portion of each deployment wire is positioned substantially within a plane that is substantially perpendicular to the longitudinal axis of the pusher wire and has an asymmetric form when viewed along a direction parallel to the longitudinal axis of the pusher wire. In the expanded state, the plurality of deployment wires are in the configuration utilised for applying the patch to an application site.

Optionally, the systems of the present disclosure comprise a steerable catheter according to the present disclosure and an internal carrier catheter as described above, wherein the self-expanding deployment structure is configured to be positioned and constrained within the lumen of the carrier catheter, which is itself configured to be positioned within the lumen of the steerable catheter. Preferably, the distal end of the carrier catheter is aligned, or is moveable to be aligned, approximately with the distal end of the steerable catheter.

The seventh and eighth aspects of the present disclosure provide methods of applying a patch to a defect site in an anatomical lumen using the systems of the fifth and sixth aspects, respectively.

In accordance with the seventh and eighth aspects of the present disclosure, the anatomical lumen may be a blood vessel and the defect site may be a cardiovascular defect, such as a tear or other lesion of the cardiovascular wall. Optionally, the cardiovascular defect may be a defect in the wall of the aorta. The defect may be located in the ascending aorta or the descending aorta. Optionally, the cardiovascular defect is an aortic dissection.

The patch is releasably attached to the self-expanding deployment structure prior to application to the defect site. The releasable attachment of the patch to the plurality of self-expanding deployment structure means that it can be released from the deployment wires when positioned and secured at the defect site. The patch can be releasably attached to the self-expanding deployment structure by a dissolvable suture/thread which dissolves on contact with an anatomical fluid (such as blood), thereby releasing the patch from the deployment structure shortly after deployment.

The ninth and tenth aspects of the present disclosure provide general methods for using the catheters of the present disclosure to deploy medical devices.

Various features of the present disclosure are described with reference to the accompanying figures, which are intended to be non-limiting.

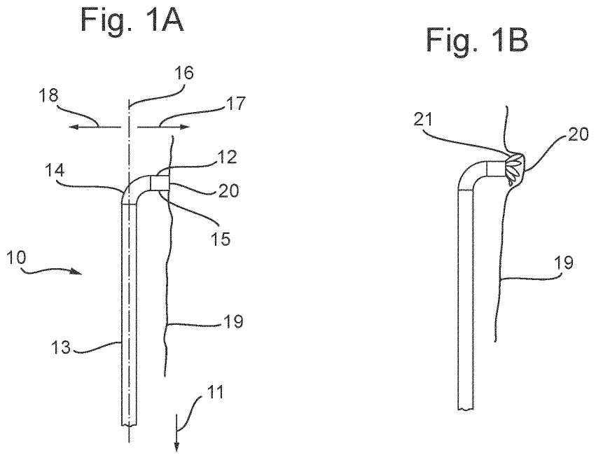

FIG. 1A is a schematic view of a prior art catheter 10. The catheter 10 comprises a proximal end 11 (not shown) and a distal end 12 comprising a distal opening. A longitudinal shaft portion 13 extends to a single steering section 14 which extends to a distal tip section 15. The longitudinal axis of the catheter is shown by a dotted line 16 and arrows 17, 18 indicate the forward and rearward directions respectively with reference to the orientation of the distal end of the catheter. The steering section 14 is shown after actuation to approximately a 90° angle. The result of the actuation is that the distal end 12 of the catheter is displaced from the longitudinal axis 16 in the forward direction 17, such that it comes into contact with the wall of an anatomical lumen 19 at a treatment site 20.

FIG. 1B shows a schematic view of the catheter of FIG. 1A during attempted advancement of a self-expanding deployment structure 21 beyond the distal end of the catheter. The self-expanding deployment structure contacts and strains against the wall of the anatomical lumen 19 causing deformation of the wall at the treatment site 20.

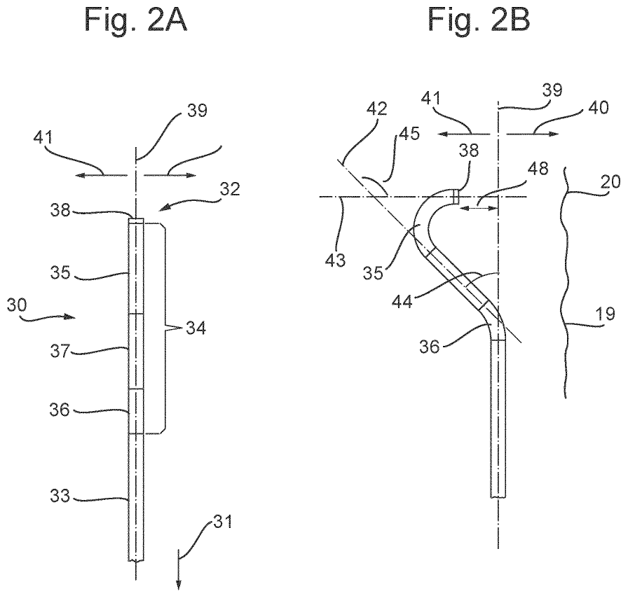

FIG. 2A shows a schematic view of a steerable catheter 30 according to the first aspect of the present disclosure in a non-actuated (linear) configuration. The steerable catheter 30 comprises a proximal end 31 (not shown) and a distal end 32 comprising a distal opening. A longitudinal shaft portion 33 extends to a steering zone 34 comprising a first steering section 35, a second steering section 36 and a connecting section 37 between the first and second steering sections. A distal tip section 38 is provided distally of the steering zone. The longitudinal axis of the catheter is shown by a dotted line 39 and arrows 40, 41 indicate the forward direction (x+ direction) and rearward direction (x− direction) respectively.

FIG. 2B shows a schematic view of the steerable catheter 30 of FIG. 2A in an actuated configuration, wherein the first steering section is actuated to steer in a first direction and the second steering section is actuated to steer in a second direction in a first plane that is parallel to and intersects the longitudinal axis 39 of the catheter. The steering angle 44 of the second steering section is defined by the longitudinal axis of the catheter 39 and the dotted line 42. The steering angle 45 of the first steering section is defined by the dotted lines 42 and 43. The distal end 38 of the catheter does not intersect with a second plane that is parallel to and intersects the longitudinal axis 39 of the catheter and is perpendicular to the first plane. Instead, the distal end 38 of the catheter is separated from the longitudinal axis by a distance 48 which represents the free space achieved by the catheter in the configuration shown.

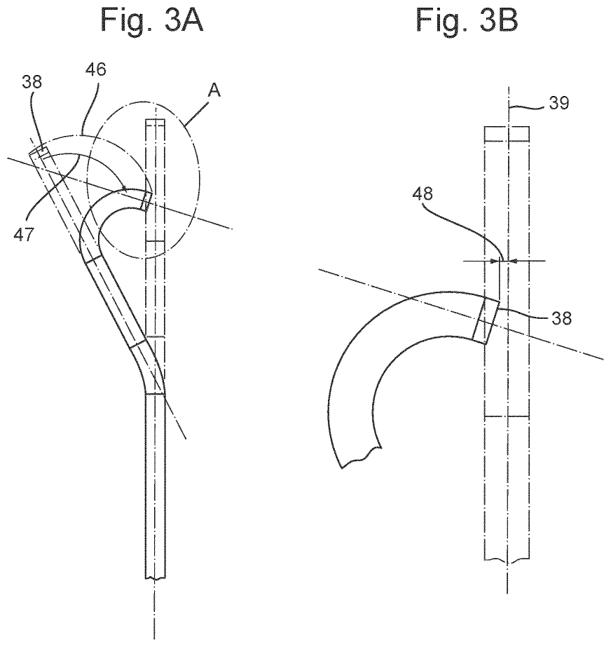

FIG. 3A shows a schematic view of the catheter of FIG. 2A with the dotted line 46 showing the arc described by the distal end 38 of the catheter upon actuation of the first steering section from 0° to its maximum steering angle 47. The arc 46 does not intersect the second plane that is parallel to and intersects the longitudinal axis 39 of the catheter and is perpendicular to the first plane.

FIG. 3B shows a close-up view of the area A of FIG. 3A. The distance 48 represents the minimum distance between the distal end 38 of the catheter and the second plane that is parallel to and intersects the longitudinal axis 39 of the catheter and is perpendicular to the first plane.

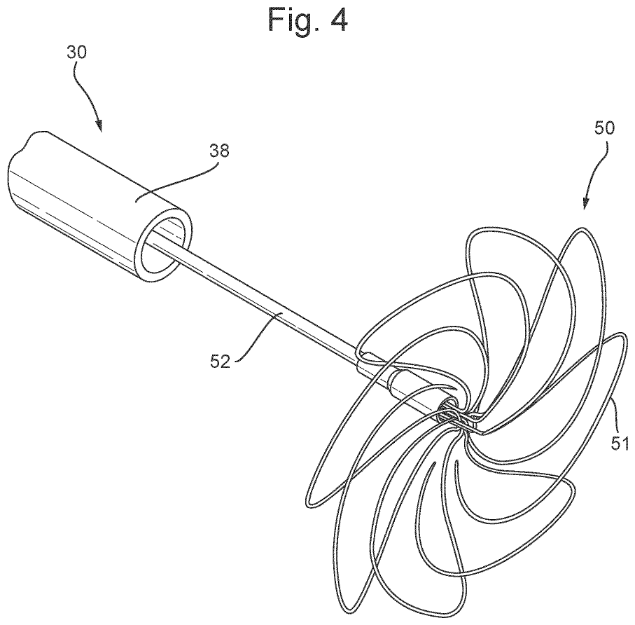

FIG. 4 shows an example of a self-expanding deployment structure 50 in an expanded state beyond the distal end 38 of the steerable catheter 30. The self-expanding deployment structure comprises a plurality of deployment wires 51 and a pusher wire 52 that is connected to the deployment wires 51 at the distal end of the pusher wire 52.

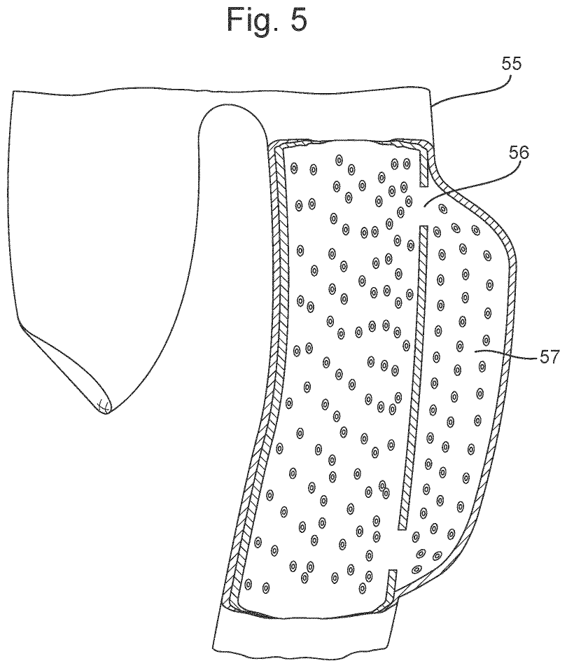

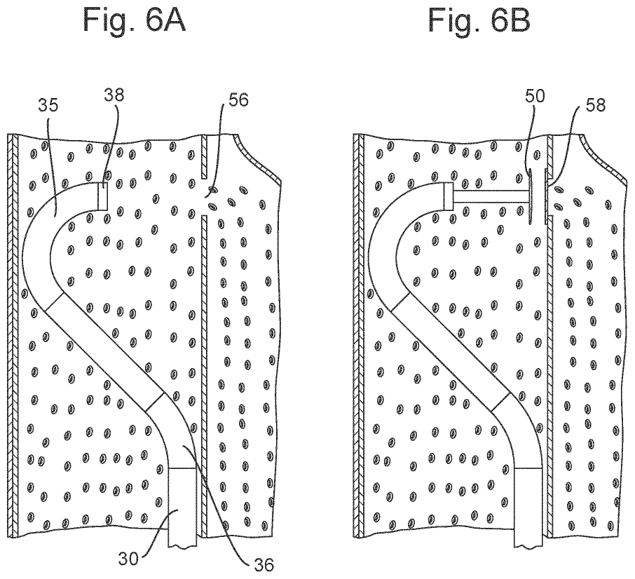

A procedure utilising the patch deployment system will now be described in relation to FIGS. 5, 6A and 6B.

FIG. 5 depicts part of the descending aorta 55 with a tear 56 in its wall. This results in blood flowing into a region known as a false lumen 57. FIG. 6A shows a steerable catheter 30 being positioned adjacent to the tear 56 in the aortic wall 55. The first and second steering sections 35, 36 are actuated to steer in opposite directions so as to create free space between the distal end 38 of the catheter and the tear 56 in the aortic wall. This free space allows for the self-expanding deployment structure 50 to be advanced from the distal end 38 of the catheter through the distal opening without causing any strain against the damaged aortic wall. FIG. 6B depicts the patch 58 that has been applied to the tear 56 by being pressed onto the tear by the self-expanding deployment structure 50, thereby blocking the flow of blood into the false lumen 57. The self-expanding deployment structure 50 can be retracted through the distal opening of the steerable catheter 30, whereupon it reassumes the unexpanded configuration. The steerable catheter 30 can then be removed from the aorta.

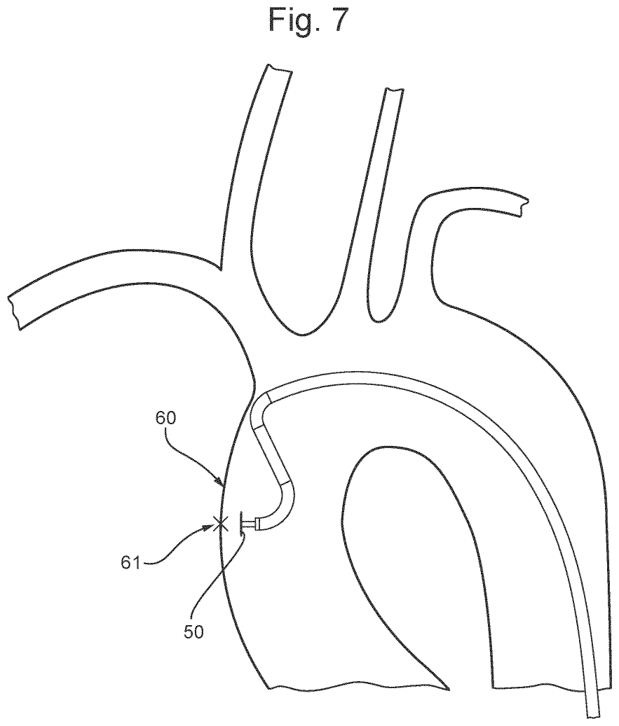

Another procedure utilising the patch deployment system of the present disclosure is described within reference to FIG. 7.

FIG. 7 depicts part of the ascending aorta 60 with a tear 61 in its wall. A steerable catheter 30 according to the present disclosure is positioned adjacent to the tear 61 by actuation of the first and second steering sections 35, 36 in opposite directions. The free space created allows for the self-expanding deployment structure 50 to be advanced from the distal end 38 of the catheter through the distal opening without causing any strain against the damaged aortic wall. FIG. 7 demonstrates how the catheter and system of the present disclosure may be used to create free space even in a complex area of the vasculature, since the steerable catheter can function simultaneously to assist the navigation of the distal end of the catheter to the treatment site and to create free space at the treatment site.

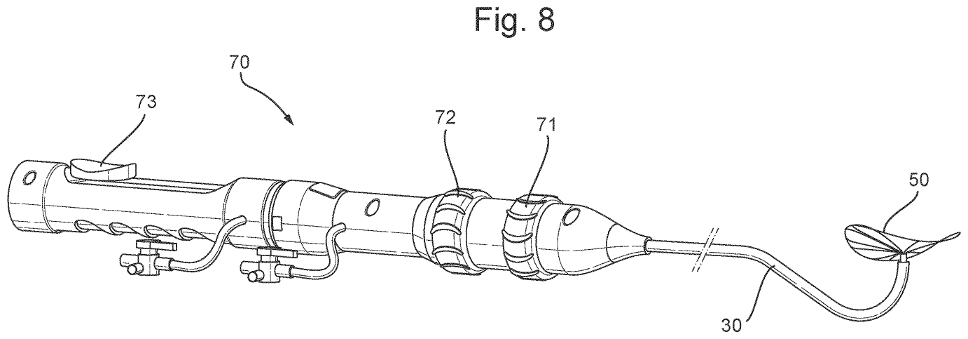

FIG. 8 depicts a patch deployment system of the present disclosure including a handle structure 70 at the proximal end of the catheter. The handle comprises a first actuator 71 for actuating the first steering section and a second actuator 72 for actuating the second steering section. The first actuator 71 is disposed distally of the second actuator 72, for correspondence with the positions of the first and second steering sections. The steerable catheter 30 functions as a guider catheter which is actively steerable to attain the correct orientation for deployment of the self-expanding deployment structure 50. An inner carrier catheter (not shown) is disposed within the lumen of the steerable catheter and the self-expanding deployment structure 50 is disposed within the carrier catheter prior to deployment.

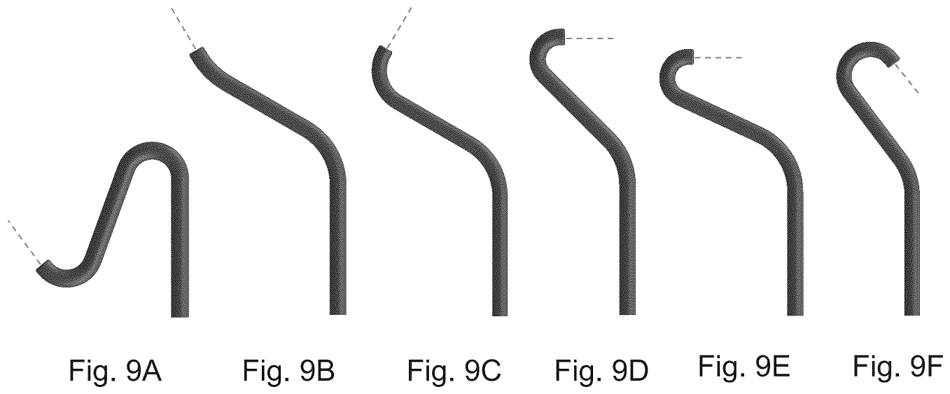

FIGS. 9A to 9F depict a catheter according to the present disclosure in various actuated configurations, demonstrating the versatility of the catheter of the present disclosure.

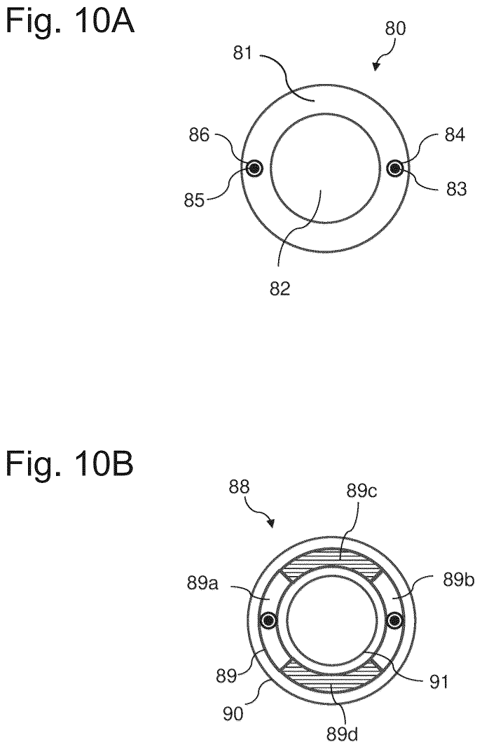

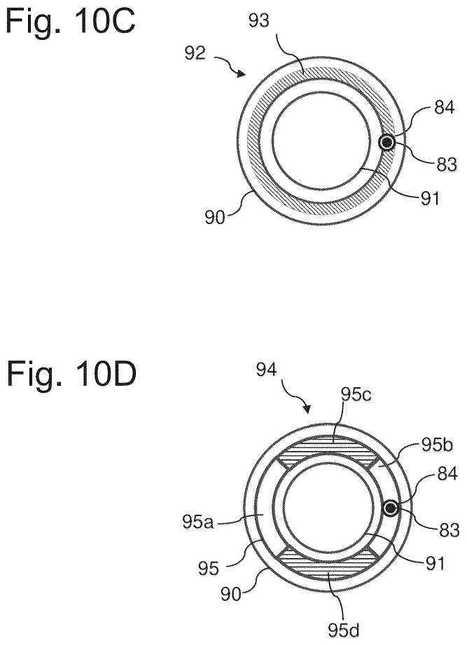

FIGS. 10A to 10D depict cross-sectional views of a catheter according to the present disclosure at various positions between the proximal and distal ends.

FIG. 10A depicts a cross-sectional view of the proximal shaft section 80 viewed from the proximal end. The proximal shaft section comprises a catheter body 81 which may comprise any suitable structure, for example a coiled pipe structure, a braided structure or a polymeric extrusion, optionally with an external polymer jacket and/or an internal liner. The catheter body 81 surrounds an internal lumen 82. Passing through the proximal shaft section 80, are a first pull wire 83 within a first pull wire lumen 84 and a second pull wire 85 within a second pull wire lumen 86.

FIG. 10B depicts a cross-sectional view of the second (proximal) steering section 88 viewed from the proximal end. The second steering section is disposed at the distal end of the proximal shaft section 80 and in the embodiment shown it comprises an articulated hypotube section 89. The lateral portions 89a, 89b of the articulated hypotube comprise flexibility enhancing features that increase the flexibility of the catheter in the steering plane (to the left and right as shown), whereas the top and bottom portions 89c, 89d of the articulated hypotube are relatively inflexible and prevent the flexion of the second steering section in the direction perpendicular to the steering plane. The first and second pull wires and their respective lumens are shown as in FIG. 10A. The distal end of the second pull wire is anchored such that tensioning of the second pull wire actuates the second steering section to steer to the left as shown. The second steering section further comprises an external jacket 90 and an internal liner 91.

FIG. 10C depicts a cross-sectional view of a connecting section 92 viewed from the proximal end. The connecting section 92 is disposed between the first and second steering sections. As shown, the connecting section comprises a hypotube portion 93 which is substantially rigid and inflexible throughout its length. Optionally, the hypotube portion 93 may be formed integrally with the articulated hypotube section 89 of the second steering section and/or the articulated hypotube section 95 of the first steering section (see below). The first pull wire 83 and the first pull wire lumen 84 extend through the connecting section to the first steering section. The connecting section further comprises an external jacket 90 and an internal liner 91.

FIG. 10D depicts a cross-sectional view of the first (distal) steering section 94 viewed from the proximal end. The first steering section is disposed at the distal end of the connecting section 92 and in the embodiment shown it comprises an articulated hypotube section 95. The lateral portions 95a, 95b of the articulated hypotube comprise flexibility enhancing features that increase the flexibility of the catheter in the steering plane (to the left and right as shown), whereas the top and bottom portions 95c, 95d of the articulated hypotube are relatively inflexible and prevent the flexion of the first steering section in the direction perpendicular to the steering plane. The first pull wire 83 and the first pull wire lumen 84 extend through the first steering section. The distal end of the first pull wire is anchored such that tensioning of the first pull wire actuates the first steering section to steer to the right as shown. The first steering section further comprises an external jacket 90 and an internal liner 91.

Optionally, the articulated hypotubes 89, 95 of the first and second steering sections and the hypotube 93 of the connecting section may be formed integrally from a single hypotube having different articulated zones corresponding to the first steering section and the second steering section and a non-articulated zone corresponding to the connecting section. Similarly, the liner 91 and/or the external jacket 90 may each be formed integrally along the entire steering zone.

The present disclosure is further described with reference to the following non-limiting embodiments:

A1 A steerable catheter comprising a longitudinal tube having a proximal end, a distal end comprising a distal opening and an internal lumen extending to the distal opening;
wherein a steering zone is provided adjacent to the distal end, wherein the steering zone comprises:
(i) a first steering section; and
(ii) a second steering section, disposed proximally of the first steering section along the longitudinal axis of the catheter;
wherein the first steering section is independently actuatable to steer in a first direction in a first plane, wherein the first plane is parallel to and intersects the longitudinal axis of the catheter;
wherein the second steering section is independently actuatable to steer in a second direction, wherein the second direction is opposite to the first direction; and
wherein the steering zone is configured such that when the second steering section is actuated to its maximum steering angle, the distal end cannot intersect a second plane, wherein the second plane is parallel to and intersects the longitudinal axis of the catheter and is perpendicular to the first plane.

A2 A steerable catheter comprising a longitudinal tube having a proximal end, a distal end comprising a distal opening and an internal lumen extending to the distal opening;
wherein a steering zone is provided adjacent to the distal end, wherein the steering zone comprises:
(i) a first steering section; and
(ii) a second steering section, disposed proximally of the first steering section along the longitudinal axis of the catheter;
wherein the first steering section is independently actuatable to steer in a first direction in a first plane, wherein the first plane is parallel to and intersects the longitudinal axis of the catheter;
wherein the second steering section is independently actuatable to steer in a second direction, wherein the second direction is opposite to the first direction; and
wherein the first steering section has a maximum steering angle of at least 60°.

A3 A steerable catheter according to embodiment A2, wherein the steering zone is configured such that when the second steering section is actuated to its maximum steering angle, the distal end cannot intersect a second plane, wherein the second plane is parallel to and intersects the longitudinal axis of the catheter and is perpendicular to the first plane.

A4 A steerable catheter according to embodiment A1 or embodiment A3, wherein the steering zone is configured such that when the second steering section is actuated to its maximum steering angle, the minimum distance between the distal end and the second plane is at least 0.1 mm, or at least 0.5 mm, or at least 1 mm, or at least 2 mm, or at least 5 mm, or at least 10 mm, or at least 15 mm, or at least 20 mm, or at least 25 mm, or at least 30 mm.

A5 A steerable catheter according to any of embodiments A1 to A4, wherein the first steering section has a maximum steering angle of at least 60°, or at least 70°, or at least 80°, or at least 90°, or at least 100°, or at least 110°, or at least 120°, or at least 130°, or at least 140°, or at least 150°, or at least 160°.

A6 A steerable catheter according to any of embodiments A1 to A5, wherein the first steering section has a maximum steering angle of no more than 270°, or no more than 240°, or at least 210°, or no more than 180°, or no more than 170°.

A7 A steerable catheter according to any of embodiments A1 to A6, wherein the second steering section has a maximum steering angle of at least 10°, or at least 20°, or at least 30°, or at least 40°, or at least 50°, or at least 60°, or at least 70°, or at least 80°, or at least 90°.

A8 A steerable catheter according to any of embodiments A1 to A7, wherein the second steering section has a maximum steering angle of no more than 210°, or no more than 180°, or no more than 150°, or no more than 120°, or no more than 110°, or no more than 100°.

A9 A steerable catheter according to any of embodiments A1 to A8, wherein the first and second steering sections are independently actuatable to orientate the distal end of the steerable catheter such that it is substantially perpendicular to the longitudinal axis of the catheter.

A10 A steerable catheter according to any of embodiments A1 to A9, wherein the maximum steering angle of the first steering section is at least 100°, or at least 110°, or at least 120°, or at least 130°, or at least 140°, or at least 150°, or at least 160° and the maximum steering angle of the second steering section is at least 10°.

A11 A steerable catheter according to any of embodiments A1 to A10, wherein the maximum steering angle of the first steering section is at least 100°, or at least 110°, or at least 120°, or at least 130°, or at least 140°, or at least 150°, or at least 160° and the maximum steering angle of the second steering section is at least 50°.

A12 A steerable catheter according to any of embodiments A1 to A11, wherein the maximum steering angle of the first steering section is at least 100°, or at least 110°, or at least 120°, or at least 130°, or at least 140°, or at least 150°, or at least 160° and the maximum steering angle of the second steering section is at least 90°.

A13 A steerable catheter according to any of embodiments A1 to A12, wherein the length of the first steering section is in the range from 5 to 180 mm, or from 10 to 180 mm, or from 20 to 150 mm, or from 30 to 120 mm, or from 40 to 100 mm, or from 45 to 90 mm, or from 50 to 80 mm.

A14 A steerable catheter according to any of embodiments A1 to A13, wherein the length of the second steering section is in the range from 5 to 180 mm, or from 10 to 180 mm, or from 20 to 150 mm, or from 30 to 120 mm, or from 40 to 100 mm, or from 45 to 90 mm, or from 50 to 80 mm.

A15 A steerable catheter according to any of embodiments A1 to A14, wherein the first steering section has a length L1 and wherein the second steering section has a length L2, wherein L1>L2.

A16 A steerable catheter according to any of embodiments A1 to A15, wherein the steering zone further comprises a substantially straight connecting section disposed between the first steering section and the second steering section.

A17 A steerable catheter according to embodiment A16, wherein the connecting section has a length L3, wherein L3 is in the range from 5 to 100 mm, or from 10 to 90 mm, or from 15 to 80 mm, or from 20 to 70 mm, or from 25 to 60 mm, or from 30 to 50 mm.

A18 A steerable catheter according to any of embodiments A1 to A17, wherein the steering zone has a total length (L1+L2+L3) in the range from 20 to 300 mm, or from 50 to 300 mm, or from 55 to 250 mm, or from 60 to 200 mm, or from 65 to 180 mm, or from 70 to 160 mm, or from 75 to 150 mm.

A19 A steerable catheter according to any of embodiments A1 to A18, further comprising a tip section extending distally from the first steering section to the distal end.

A20 A steerable catheter according to embodiment A19, wherein the tip section has a length L4, wherein L4 is no more than 50 mm, or no more than 40 mm, or no more than 30 mm, or no more than 20 mm, or no more than 10 mm, or no more than 8 mm, or no more than 6 mm, or no more than 5 mm, or no more than 4 mm, or no more than 2 mm.

A21 A steerable catheter according to any of embodiments A1 to A20, wherein the outer diameter of the steering zone is no more than 20 Fr, or no more than 18 Fr, or no more than 16 Fr, or no more than 14 Fr, or no more than 12 Fr, or no more than 10 Fr.

A22 A steerable catheter according to any of embodiments A1 to A21, comprising first and second pull wires configured to actuate each of the first and second steering sections, respectively.

A23 A steerable catheter according to any of embodiments A1 to A22, comprising a handle portion at the proximal end, the handle portion comprising first and second actuators respectively configured to actuate each of the first and second steering sections.

A24 A steerable catheter according to any of embodiments A1 to A23, further comprising a carrier catheter extending through the steerable catheter, wherein the carrier catheter is axially movable with respect to the steerable catheter.

A25. A steerable catheter according to any of embodiments A1 to A24, wherein the first steering section has lower resistance to bending in the first plane (x,y-plane) than in the second plane (y,z-plane) and the second steering section preferably has lower resistance to bending in the steering plane than in the direction perpendicular to the steering plane.

A26. A steerable catheter according to embodiment A25, wherein the first steering section is flexible in both directions in the first plane and is relatively inflexible, or even non-flexible, in the second plane (y,z-plane, in both the z− and z+ directions).

A27. A steerable catheter according to embodiment A25, wherein the first steering section is flexible in the forward direction (x+ direction) in the first plane, and is relatively inflexible, or even non-flexible, in both the rearward direction (x− direction) and the second plane (y,z-plane, in both the z− and z+ directions).

A28. A steerable catheter according to any of embodiments A25 to A27, wherein the second steering section is flexible in both directions in the steering plane (forward direction and rearward direction) and is relatively inflexible, or even non-flexible, in the direction perpendicular to the steering plane.

A29. A steerable catheter according to any of embodiments A25 to A27, wherein the second steering section is flexible in the rearward direction in the steering plane, and relatively inflexible, or even non-flexible, in the both the forward direction in the steering plane and in the direction perpendicular to the steering plane.

A30. A steerable catheter according to any of embodiments A25 to A29, wherein the steering plane of the second steering section is the first plane.

A31. A steerable catheter according to any of embodiments A25 to A30, wherein the first and second steering sections each comprise an articulated hypotube section.

A32. A steerable catheter according to embodiment A31, wherein the steering zone is integrally formed from a single hypotube comprising first and second articulated zones and an optional non-articulated zone between the first and second articulated zones.

A33. A steerable catheter according to any of embodiments A1 to A32, further comprising a proximal shaft section extending from the proximal end to the steering zone.

A34. A steerable catheter according to embodiment A33, wherein the ratio of the internal lumen diameter to the external diameter of the catheter in the proximal shaft section is at least 0.60, or at least 0.65, or at least 0.70, or at least 0.75, or at least 0.80, or at least 0.85, or at least 0.90.

A35. A steerable catheter according to embodiment A34, wherein the ratio of the internal lumen diameter to the external diameter of the catheter is at least 0.60, or at least 0.65, or at least 0.70, or at least 0.75, or at least 0.80, or at least 0.85, or at least 0.90 throughout the proximal shaft section and the steering zone.

A36. A system comprising a steerable catheter according to any of embodiments A1 to A35 and a carrier catheter extending through the steerable catheter, wherein the carrier catheter is axially movable with respect to the steerable catheter.

B1 A steerable catheter comprising a longitudinal tube having a proximal end, a distal end comprising a distal opening and an internal lumen extending to the distal opening;
wherein a steering zone is provided adjacent to the distal end, wherein the steering zone comprises:
(i) a first steering section, wherein the first steering section is independently actuatable to steer in a first direction in a first plane, wherein the first plane is parallel to and intersects the longitudinal axis of the catheter; and
(ii) a shape-set curve steering section, disposed proximally of the first steering section along the longitudinal axis of the catheter, wherein the shape set curve steering section curves in a second direction and is independently actuatable to steer in the first direction, wherein the second direction is opposite to the first direction; and
wherein the steering zone is configured such that, when the shape-set curve steering section is not actuated, the distal end of the catheter cannot intersect a second plane, wherein the second plane is parallel to and intersects the longitudinal axis of the catheter and is perpendicular to the first plane.

B2 A steerable catheter comprising a longitudinal tube having a proximal end, a distal end comprising a distal opening and an internal lumen extending to the distal opening;
wherein a steering zone is provided adjacent to the distal end, wherein the steering zone comprises:
(i) a first steering section, wherein the first steering section is independently actuatable to steer in a first direction in a first plane, wherein the first plane is parallel to and intersects the longitudinal axis of the catheter; and
(ii) a shape-set curve steering section, disposed proximally of the first steering section along the longitudinal axis of the catheter, wherein the shape set curve steering section curves in a second direction in the first plane and is independently actuatable to steer in the first direction in the first plane, wherein the second direction is opposite to the first direction; and
wherein the first steering section has a maximum steering angle of at least 60°.

B3 A steerable catheter according to embodiment B2, wherein the steering zone is configured such that the distal end cannot intersect a second plane, wherein the second plane is parallel to and intersects the longitudinal axis of the catheter and is perpendicular to the first plane.

B4 A steerable catheter according to embodiment B1 or embodiment B3, wherein the steering zone is configured such that the minimum distance between the distal end and the second plane is at least 0.1 mm, or at least 0.5 mm, or at least 1 mm, or at least 2 mm, or at least 5 mm, or at least 10 mm, or at least 15 mm, or at least 20 mm, or at least 25 mm, or at least 30 mm.

B5 A steerable catheter according to any of embodiments B1 to B4, wherein the first steering section has a maximum steering angle of at least 60°, or at least 70°, or at least 80°, or at least 90°, or at least 100°, or at least 110°, or at least 120°, or at least 130°, or at least 140°, or at least 150°, or at least 160°.

B6 A steerable catheter according to any of embodiments B1 to B5, wherein the first steering section has a maximum steering angle of no more than 270°, or no more than 240°, or no more than 210°, or no more than 180°, or no more than 170°.

B7 A steerable catheter according to any of embodiments B1 to B6, wherein the shape-set curve steering section has a shape-set curve angle in the range from 10° to 210°, or from 10° to 180°, or from 10° to 150°, or from 10° to 120°, or from 10° to 90°, or from 15° to 60°, or from 20° to 45°.

B8 A steerable catheter according to any of embodiments B1 to B7, wherein the first steering section is actuatable to orientate the distal end of the steerable catheter such that it is substantially perpendicular to the longitudinal axis of the catheter.

B9 A steerable catheter according to any of embodiments B1 to B8, wherein the maximum steering angle of the first steering section is at least 100°, or at least 110°, or at least 120°, or at least 130°, or at least 140°, or at least 150°, or at least 160° and the shape-set curve angle of the shape-set curve steering section is at least 10°.

B10 A steerable catheter according to any of embodiments B1 to B9, wherein the maximum steering angle of the first steering section is at least 100°, or at least 110°, or at least 120°, or at least 130°, or at least 140°, or at least 150°, or at least 160° and the shape-set curve angle of the shape-set curve steering section is at least 15°.

B11 A steerable catheter according to any of embodiments B1 to B10, wherein the maximum steering angle of the first steering section is at least 100°, or at least 110°, or at least 120°, or at least 130°, or at least 140°, or at least 150°, or at least 160° and the shape-set curve angle of the shape-set curve steering section is at least 30°.

B12 A steerable catheter according to any of embodiments B1 to B11, wherein the length of the first steering section is in the range from 5 to 180 mm, or from 10 to 180 mm, or from 20 to 150 mm, or from 30 to 120 mm, or from 40 to 100 mm, or from 45 to 90 mm, or from 50 to 80 mm.

B13 A steerable catheter according to any of embodiments B1 to B12, wherein the length of the shape-set curve steering section is in the range from 5 to 180 mm, or from 10 to 180 mm, or from 20 to 150 mm, or from 30 to 120 mm, or from 40 to 100 mm, or from 45 to 90 mm, or from 50 to 80 mm.

B14 A steerable catheter according to any of embodiments B1 to B13, wherein the first steering section has a length L1 and wherein the shape-set curve steering section has a length L5, wherein L1>L5.

B15 A steerable catheter according to any of embodiments B1 to B14, wherein the steering zone further comprises a substantially straight connecting section disposed between the first steering section and the shape-set curve steering section.

B16 A steerable catheter according to embodiment B15, wherein the connecting section has a length L3, wherein L3 is in the range from 5 to 100 mm, or from 10 to 90 mm, or from 15 to 80 mm, or from 20 to 70 mm, or from 25 to 60 mm, or from 30 to 50 mm.

B17 A steerable catheter according to any of embodiments B1 to B16, wherein the steering zone has a total length (L1+L5+L3) in the range from 20 to 300 mm, or from 50 to 300 mm, or from 55 to 250 mm, or from 60 to 200 mm, or from 65 to 180 mm, or from 70 to 160 mm, or from 75 to 150 mm.

B18 A steerable catheter according to any of embodiments B1 to B17, further comprising a tip section extending distally from the first steering section to the distal end.

B19 A steerable catheter according to embodiment B18, wherein the tip section has a length L4, wherein L4 is no more than 50 mm, or no more than 40 mm, or no more than 30 mm, or no more than 20 mm, or no more than 10 mm, or no more than 8 mm, or no more than 6 mm, or no more than 5 mm, or no more than 4 mm, or no more than 2 mm.

B20 A steerable catheter according to any of embodiments B1 to B19, wherein the outer diameter of the steering zone is no more than 20 Fr, or no more than 18 Fr, or no more than 16 Fr, or no more than 14 Fr, or no more than 12 Fr, or no more than 10 Fr.

B21 A steerable catheter according to any of embodiments B1 to B20, comprising a first pull wire configured to actuate the first steering section.

B22 A steerable catheter according to any of embodiments B1 to B21, comprising a handle portion at the proximal end, the handle portion comprising a first actuator configured to actuate the first steering section.

B23 A steerable catheter according to any of embodiments B1 to B22, further comprising a carrier catheter extending through the steerable catheter, wherein the carrier catheter is axially movable with respect to the steerable catheter.

B24. A steerable catheter according to any of embodiments B1 to B23, wherein the first steering section has lower resistance to bending in the first plane (x,y-plane) than in the second plane (y,z-plane).

B25. A steerable catheter according to embodiment B24, wherein the first steering section is flexible in both directions in the first plane and is relatively inflexible, or even non-flexible, in the second plane (y,z-plane, in both the z− and z+ directions).

B26. A steerable catheter according to embodiment B24, wherein the first steering section is flexible in the forward direction (x+ direction) in the first plane, and is relatively inflexible, or even non-flexible, in both the rearward direction (x− direction) and the second plane (y,z-plane, in both the z− and z+ directions).

B27. A steerable catheter according to any of embodiments B24 to B26, wherein the shape-set curve steering section has lower resistance to bending in the steering plane than in the direction perpendicular to the steering plane.

B28. A steerable catheter according to any of embodiments B1 to B27, further comprising a proximal shaft section extending from the proximal end to the steering zone.

B29. A steerable catheter according to embodiment B28, wherein the ratio of the internal lumen diameter to the external diameter of the catheter in the proximal shaft section is at least 0.60, or at least 0.65, or at least 0.70, or at least 0.75, or at least 0.80, or at least 0.85, or at least 0.90.

B30. A steerable catheter according to embodiment B29, wherein the ratio of the internal lumen diameter to the external diameter of the catheter is at least 0.60, or at least 0.65, or at least 0.70, or at least 0.75, or at least 0.80, or at least 0.85, or at least 0.90 throughout the proximal shaft section and the steering zone.

B31. A system comprising a steerable catheter according to any of embodiments B1 to B30, and a carrier catheter extending through the steerable catheter, wherein the carrier catheter is axially movable with respect to the steerable catheter.

C1 A system for deploying a patch to an anatomical lumen, the system comprising:
(a) a steerable catheter as defined in any of embodiments A1 to A35; and
(b) a deployment device, the deployment device comprising:
   (i) a self-expanding deployment structure configured to be in an unexpanded state when positioned and constrained within the lumen of the steerable catheter, and configured to self-expand into an expanded state when displaced through the distal opening and beyond the distal end of the catheter; and
   (ii) a pusher wire that is axially moveable within the lumen of the catheter to move the self-expanding deployment structure between an unexpanded state within the catheter and an expanded state beyond the distal end of the catheter.

C2 A system according to embodiment C1, wherein the self-expanding deployment structure comprises a plurality of deployment wires, wherein each deployment wire:
(i) has a first end and a second end, and the first end and the second end are connected to the distal end of the pusher wire; and
(ii) is configured to be in an unexpanded state when positioned and constrained within a catheter, and is configured to self-expand into an expanded state when positioned beyond a distal end of the catheter and not constrained, wherein in the expanded state at least a portion of the wire is positioned substantially within a plane that is substantially perpendicular to the longitudinal axis of the pusher wire.

C3 A system according to embodiment C1 or C2, wherein each deployment wire has an asymmetric form when viewed along a direction parallel to the longitudinal axis of the pusher wire.

C4 A system according to any of embodiments C1 to C3, further comprising a patch releasably attached to the self-expanding deployment structure.

C5 A system according to embodiment C4, wherein the patch is a cardiovascular repair patch.

C6 A system according to any of embodiments C1 to C5, further comprising a carrier catheter, wherein the carrier catheter is configured to be positioned and axially movable within the lumen of the steerable catheter, and wherein the self-expanding deployment structure is configured to be positioned and constrained within the lumen of the carrier catheter.

D1 A system for deploying a patch to an anatomical lumen, the system comprising:
(a) a steerable catheter as defined in any of embodiments B1 to B24; and
(b) a deployment device, the deployment device comprising:
   (i) a self-expanding deployment structure configured to be in an unexpanded state when positioned and constrained within the lumen of the steerable catheter, and configured to self-expand into an expanded state when displaced through the distal opening and beyond the distal end of the catheter; and
   (ii) a pusher wire that is axially moveable within the lumen of the catheter to move the self-expanding deployment structure between an unexpanded state within the catheter and an expanded state beyond the distal end of the catheter.

D2 A system according to embodiment D1, wherein the self-expanding deployment structure comprises a plurality of deployment wires, wherein each deployment wire:
(i) has a first end and a second end, and the first end and the second end are connected to the distal end of the pusher wire; and
(ii) is configured to be in an unexpanded state when positioned and constrained within a catheter, and is configured to self-expand into an expanded state when positioned beyond a distal end of the catheter and not constrained, wherein in the expanded state at least a portion of the wire is positioned substantially within a plane that is substantially perpendicular to the longitudinal axis of the pusher wire.

D3 A system according to embodiment D1 or D2, wherein each deployment wire has an asymmetric form when viewed along a direction parallel to the longitudinal axis of the pusher wire.

D4 A system according to any of embodiments D1 to D3, further comprising a patch releasably attached to the self-expanding deployment structure.

D5 A system according to embodiment D4, wherein the patch is a cardiovascular repair patch.

D6 A system according to any of embodiments D1 to D5, further comprising a carrier catheter, wherein the carrier catheter is configured to be positioned and axially movable within the lumen of the steerable catheter, and wherein the self-expanding deployment structure is configured to be positioned and constrained within the lumen of the carrier catheter.

E1 A method of applying a patch to a defect site in an anatomical lumen, the method comprising:
(i) providing a system as defined in embodiment C4 or C5, with the self-expanding deployment structure positioned and constrained in an unexpanded state within the lumen of the catheter and further comprising a patch releasably attached to the self-expanding patch deployment structure;
(ii) inserting the catheter into the anatomical lumen so as to position the distal end of the catheter adjacent to the defect site;
(iii) actuating the first and second steering sections, to steer the first steering section in the first direction in the first plane and to steer the second steering section in the second direction in the first plane, such that the distal end of the steerable catheter is oriented toward the defect site;
(iv) advancing the pusher wire to move the self-expanding patch deployment structure into an expanded state beyond the distal end of the catheter;
(v) applying the patch to the defect site;
(v) releasing the patch from the self-expanding patch deployment structure;
(vi) withdrawing the pusher wire to retract the self-expanding patch deployment structure into the unexpanded state within the lumen of the catheter; and
(vii) removing the catheter from the anatomical lumen.

E2 A method according to embodiment E1, wherein the anatomical lumen is a blood vessel and wherein the defect site is a cardiovascular defect.

E3 A method according to embodiment E2, wherein the cardiovascular defect is a defect in the wall of the aorta.

E4 A method according to embodiment E3, wherein the cardiovascular defect is an aortic dissection.

E5 A method according to embodiment E1, wherein the defect site is a cardiac defect.

F1 A method of applying a patch to a defect site in an anatomical lumen, the method comprising:
(i) providing a system as defined in embodiment D4 or D5, with the self-expanding deployment structure positioned and constrained in an unexpanded state within the lumen of the catheter and further comprising a patch releasably attached to the self-expanding patch deployment structure;
(ii) inserting the catheter into the anatomical lumen while actuating the shape-set curve steering section so as to position the distal end of the catheter adjacent to the defect site;
(iii) actuating the first steering sections, to steer the first steering section in the first direction in the first plane and releasing the actuation of the shape-set curve steering section, such that the distal end of the steerable catheter is oriented toward the defect site;
(iv) advancing the pusher wire to move the self-expanding patch deployment structure into an expanded state beyond the distal end of the catheter;
(v) applying the patch to the defect site;
(v) releasing the patch from the self-expanding patch deployment structure;
(vi) withdrawing the pusher wire to retract the self-expanding patch deployment structure into the unexpanded state within the lumen of the catheter; and
(vii) removing the catheter from the anatomical lumen.

F2 A method according to embodiment F1, wherein the anatomical lumen is a cardiovascular lumen and wherein the defect site is a cardiovascular defect.

F3 A method according to embodiment F2, wherein the cardiovascular defect is a defect in the wall of the aorta.

F4 A method according to embodiment F3, wherein the cardiovascular defect is an aortic dissection.

F5 A method according to embodiment F1, wherein the defect site is a cardiac defect.

G1. A method of deploying a medical device using a catheter a steerable catheter as defined in any of embodiments A1 to A35, the method comprising:
(i) inserting the catheter into an anatomical lumen adjacent to a treatment site;
(ii) actuating the first and second steering sections, to steer the first steering section in the first direction in the first plane and to steer the second steering section in the second direction in the first plane, such that the distal end of the catheter is oriented toward the defect site; and
(iii) deploying the medical device via the distal opening.

H1. A method of deploying a medical device using a catheter as defined in any of embodiments B1 to B24, the method comprising:
(i) inserting the catheter into an anatomical lumen while actuating the shape-set curve steering section so as to position the distal opening of the catheter adjacent to a treatment site;
(ii) actuating the first steering section, to steer the first steering section in the first direction in the first plane, and releasing the actuation of the shape-set curve steering section such that the distal opening of the steerable catheter is oriented toward the defect site; and
(iii) deploying the medical device via the distal opening.

What is claimed is:

1. A steerable catheter comprising a longitudinal tube having a proximal end, a distal end comprising a distal opening and an internal lumen extending to the distal opening;
wherein a steering zone is provided adjacent to the distal end, wherein the steering zone comprises:
a first steering section; and
a second steering section, disposed proximally of the first steering section along a longitudinal axis of the steerable catheter;
wherein the first steering section is independently actuatable to steer in a first direction in a first plane, wherein the first plane is parallel to and intersects the longitudinal axis of the steerable catheter;
wherein the second steering section is independently actuatable to steer in a second direction, wherein the second direction is opposite to the first direction; and
wherein the steering zone is configured such that when the second steering section is actuated to its maximum steering angle, the distal end cannot intersect a second plane, wherein the second plane is parallel to and intersects the longitudinal axis of the steerable catheter and is perpendicular to the first plane.

2. The steerable catheter according to claim 1, wherein the steering zone is configured such that when the second steering section is actuated to its maximum steering angle, a minimum distance between the distal end and the second plane is at least 0.1 mm.

3. The steerable catheter according to claim 1, wherein the first steering section has a maximum steering angle of at least 160°.

4. The steerable catheter according to claim 1, wherein the first steering section has a maximum steering angle of no more than 270°.

5. The steerable catheter according to claim 1, wherein the second steering section has a maximum steering angle of at least 90°.

6. The steerable catheter according to claim 1, wherein the second steering section has a maximum steering angle of no more than 210°.

7. The steerable catheter according to claim 1, wherein the first and second steering sections are independently actuatable to orientate the distal end of the steerable catheter such that it is substantially perpendicular to the longitudinal axis of the catheter.

8. The steerable catheter according to claim 1, wherein a maximum steering angle of the first steering section is at least 160° and the maximum steering angle of the second steering section is at least 10°.

9. The steerable catheter according to claim 1, wherein a maximum steering angle of the first steering section is at least 160° and the maximum steering angle of the second steering section is at least 50°.

10. The steerable catheter according to claim 1, wherein a length of the first steering section is in a range from 5 to 180 mm.

11. The steerable catheter according to claim 1, wherein a length of the second steering section is in a range from 5 to 180 mm.

12. The steerable catheter according to claim 1, wherein the first steering section has a length L1 and wherein the second steering section has a length L2, wherein L1>L2.

13. The steerable catheter according to claim 1, wherein the steering zone further comprises a substantially straight connecting section disposed between the first steering section and the second steering section, optionally wherein the connecting section has a length L3, wherein L3 is in a range from 5 to 100 mm.

14. The steerable catheter according to claim 1, wherein the steering zone has a total length in a range from 20 to 300 mm.

15. The steerable catheter according to claim 1, further comprising a tip section extending distally from the first steering section to the distal end, optionally wherein the tip section has a length L4, wherein L4 is no more than 50 mm.

16. The steerable catheter according to claim 1, comprising first and second pull wires configured to actuate each of the first and second steering sections, respectively.

17. The steerable catheter according to claim 1, wherein the first steering section has lower resistance to bending in the first plane than in the second plane and/or wherein the second steering section has lower resistance to bending in a steering plane than in a direction perpendicular to the steering plane.

18. The steerable catheter according to claim 17, wherein the first steering section is flexible in both directions in the first plane and is relatively inflexible or non-flexible in the second plane.

19. The steerable catheter according to claim 17, wherein the first steering section is flexible in a forward direction in the first plane, and is relatively inflexible or non-flexible in both a rearward direction and the second plane.

20. The steerable catheter according to claim 17, wherein the second steering section is flexible in a forward direction and a rearward direction in the steering plane and is relatively inflexible or non-flexible in a direction perpendicular to the steering plane.

21. The steerable catheter according to claim 17, wherein the second steering section is flexible in a rearward direction in the steering plane, and relatively inflexible or non-flexible in both a forward direction in the steering plane and in a direction perpendicular to the steering plane.

22. The steerable catheter according to claim 17, wherein the steering plane of the second steering section is the first plane.

23. The steerable catheter according to claim 17, wherein the first and second steering sections each comprise an articulated hypotube section.

24. The steerable catheter according to claim 23, wherein the steering zone is integrally formed from a single hypotube comprising first and second articulated zones and an optional non-articulated zone between the first and second articulated zones.

25. The steerable catheter according to claim 1, further comprising a proximal shaft section extending from the proximal end to the steering zone.

26. The steerable catheter according to claim 25, wherein a ratio of an internal lumen diameter to an external diameter of the catheter in the proximal shaft section is at least 0.60.

27. The steerable catheter according to claim 26, wherein the ratio of the internal lumen diameter to the external diameter of the catheter is at least 0.60 throughout the proximal shaft section and the steering zone.

28. A steerable catheter comprising a longitudinal tube having a proximal end, a distal end comprising a distal opening, and an internal lumen extending to the distal opening;
wherein a steering zone is provided adjacent to the distal end, wherein the steering zone comprises:
a first steering section, wherein the first steering section is independently actuatable to steer in a first direction in a first plane, wherein the first plane is parallel to and intersects a longitudinal axis of the steerable catheter; and
a shape-set curve steering section, disposed proximally of the first steering section along the longitudinal axis of the steerable catheter, wherein the shape-set curve steering section is curved in a second direction and is independently actuatable to steer in the first direction, wherein the second direction is opposite to the first direction; and
wherein the steering zone is configured such that when the shape-set curve steering section is not actuated, the distal end of the steerable catheter cannot intersect a second plane, wherein the second plane is parallel to and intersects the longitudinal axis of the steerable catheter and is perpendicular to the first plane.

29. The steerable catheter according to claim 28, further comprising a carrier catheter extending through the steerable catheter, wherein the carrier catheter is axially movable with respect to the steerable catheter.

30. A system for deploying a patch to an anatomical lumen, the system comprising:
the steerable catheter as defined in claim 1; and
a deployment device, the deployment device comprising:
a self-expanding deployment structure configured to be in an unexpanded state when positioned and constrained within the anatomical lumen of the steerable catheter, and configured to self-expand into an expanded state when displaced through the distal opening and beyond the distal end of the catheter; and
a pusher wire that is axially moveable within the anatomical lumen of the catheter to move the self-expanding deployment structure between the unexpanded state within the catheter and the expanded state beyond the distal end of the catheter.
